(19) United States
(12) Patent Application Publication
(10) Pub. No.: US 2009/0118641 A1
(43) Pub. Date: May 7, 2009

(54) Devices, Methods, and Kits for a Biopsy Device

(76) Inventors: Jacques Van Dam, San Carlos, CA (US); James S. Cybulski, Menlo Park, CA (US); Venkata Gurukula, Fremont, CA (US)

Correspondence Address: WILSON SONSINI GOODRICH & ROSATI, 650 PAGE MILL ROAD, PALO ALTO, CA, 94304-1050, US

(21) Appl. No.: 12/166,813

(22) Filed: Jul. 2, 2008

Related U.S. Application Data
(60) Provisional application No. 60/984,997, filed on Nov. 2, 2007.

(51) Int. Cl. A61B 10/02

(52) U.S. Cl. 600/567

(57) ABSTRACT

Provided herein are biopsy devices each comprised of a tissue collection element having a distal end and a proximal end connected to a drive mechanism. In one embodiment, the tissue collection element can be formed from a material having a first constrained configuration when positioned within the outer needle prior to deployment and a second unconstrained configuration when extended distally beyond the distal end of the outer needle. In another embodiment, the tissue collection element can be formed from a material having a first constrained configuration when a stylet is inserted into the tissue collection element and a second unconstrained configuration when the stylet is retracted from within the tissue collection element. In a third embodiment, the tissue collection element comprises a helical cutting edge along at least a portion of the length of the tissue collection element, wherein the helical cutting edge is adaptable to cut a portion of tissue from the target location. The tissue collection element is translationally and rotationally moveable within a target location in response to actuation by the drive mechanism, thereby collecting tissue. The biopsy devices provided herein can further comprise provisions for echogenecity, a non-friction coating at the tip, and means for providing aspiration. Further provided herein are methods for using the devices described and a kit.

### CROSS-REFERENCE

This application claims the benefit of U.S. Provisional Application No. 60/984,997, filed Nov. 2, 2007, which application is incorporated herein by reference.

### BACKGROUND OF THE INVENTION

Fine needle aspiration (FNA) has been a well-accepted method for obtaining tissue samples for pathologic or histologic analysis in diagnosing tumors of the pancreas and other soft tissue organs. Endoscopic ultrasound (EUS) and EUS-guided fine needle aspiration (EUS-FNA) have become important tools in the evaluation of pancreatic masses.

Conventional surgical techniques for obtaining tissue samples accessible only through a flexible ultrasound-endoscope using a fine needle generally require numerous needle sticks. These procedures often result in obtaining a small number of cells with each aspiration, cells which may or may not be diagnostic. In addition, such procedures are often traumatic because of the multiple needle passes that it necessitates. This is especially true in the case of pancreatic biopsies. The pancreas secretes digestive enzymes. When injured, these enzymes are released, and may induce self digestion, and necrosis of the pancreas, and adjacent organs. The current technique used during Endoscopic Ultrasound Fine Needle Aspiration (EUS-FNA) of a pancreatic tumor entails the passage of a 19-25 gauge stainless steel needle. This needle is passed through the working channel of a linear echo endoscope under real-time guidance into the endo-sonographically visualized pancreatic mass. The needle is moved back and forth multiple times through the lesion with varying degrees of suction applied to it. The specimens obtained are then deposited onto a cytology slide for immediate fixation, staining and cytopathologic examination.

Aspirating a sample from a fluid medium through a needle is a simple procedure. Aspirating a sample from a solid mass is difficult. Most pancreatic EUS-FNA procedures take up to 30 needle passes to make a definitive cytological diagnosis of pancreatic carcinoma. Oftentimes, the only cells that are obtained are blood cells, or normal pancreatic tissue cells. Even when tumor cells are captured, these are often fragmented, and separated from each other. It is therefore almost impossible to differentiate a primary pancreatic tumor from a metastatic lesion.

Despite the time consuming and traumatic nature of the current FNA procedure, the consequence of a non-diagnostic aspirate is worse, because a missed diagnosis of pancreatic cancer is a sure death sentence. Therefore, if a pancreatic tumor is suspected but the FNA result is negative, the patient must then undergo a pancreatic biopsy through an abdominal incision. Although needles for taking core biopsies of internal organs exist, these needles are much thicker than the needles used during fine tissue aspiration. An example of such a needle is the Mangini needle, with which percutanous liver biopsies are used. In order to introduce this needle into the liver, an incision must be made in the skin with the sharp tip of a scalpel. The needle is then pushed into the incision, and under aspiration is quickly pushed in and out of the liver with a quick stabbing motion. The resulting core biopsy is almost always diagnostic, and ample to examine sheets of tissue cells representative of the pathology that is sought. The injury, however, is much greater than that inflicted with a fine needle.

The choices for obtaining diagnostic tissue from internal organs are three fold. The first choice is to obtain a biopsy though an open operative incision or a laparoscopic technique, which entails surgical intervention. The second option is to use a large diameter stiff stainless steel needle. This method may only be used for lesions that are near the exterior of the body, such as described above in relation to the Mangini needle. The third method is to obtain cells through a fine needle with ultrasound guidance. While this method is least traumatic with only one needle introduction, it produces a poor yield of diagnostic material. In the best case scenario, and after multiple needle sticks, several cells of the tumor are retrieved. Because the cells are obtained separate from one another, they are examined by the pathologist without their spatial relationship to the rest of the organ that they originated front In the worst case, even these tumor cells are not obtained, only blood cells and normal tissue, necessitating one of the more invasive procedures. It is therefore most desirable to have an instrument of being passed through the flexible endoscope that is both delicate so as not to traumatize the area that is being biopsied, and at the same time be capable of obtaining a core tissue biopsy that will be diagnostic. It would be of great advantage if diagnostic certainty could be achieved with a minimal number of instrument passes, thus achieving excellent results with minimal trauma to the patient.

The fine needle aspiration technique is also widely used to obtain cells from suspected lesions in organs that are more superficial. These organs include breast, prostate, thyroid and parathyroid. Although these organs are more accessible to the needle than the pancreas, the trauma incurred by a thick core biopsy needle stick is great. Millions of people undergo fine needle aspirations for suspected cancer. Here too, 10-15 needle sticks are required to obtain what is deemed a sufficient number of cells for an adequate specimen. A device for obtaining a measurable tissue sample in one extraction would be highly beneficial for biopsy.

### SUMMARY OF THE INVENTION

Provided herein is a biopsy device comprising: an outer needle having a distal end and a proximal end connected to a drive mechanism; and a tissue collection element having a distal end and a proximal end, the tissue collection element formed from material having a first constrained configuration when positioned within the outer needle prior to deployment and a second unconstrained configuration when extended distally beyond the distal end of the outer needle, wherein the tissue collection element is translationally and rotationally moveable within the outer needle and distally beyond the distal end of the outer needle in response to actuation by the drive mechanism. The distal end of the tissue collection element can deviate from the central axis of the outer needle from 0 degrees to 180 degrees and rotates around the central axis of the outer needle from 0 degrees to about 360 degrees. The distal end of the tissue collection element can deviate from a central axis of the outer needle along an angle, radius, helical path, or contour. The distal end of the tissue collection element can comprise an opening, wherein the opening obtains a portion of tissue having a cross-sectional diameter greater than the cross-sectional diameter of the outer needle. The tissue collection element can be translated from within the outer needle to a target location. In some embodiments, the distal end of the tissue collection element is rotationally actuated to produce a rotational motion. Furthermore, the rotational motion can be a motion selected from the group consisting of continuous, intermittent, reciprocating, and combinations thereof. In some embodiments, the tissue collection element is adaptable to be moved manually. Alternatively, the tissue collection element is adaptable to be moved automatically or semi-automatically. Additionally, the outer needle of the biopsy device can be adaptable to or adapted and configured to be moved manually. Alternatively, the outer needle can be adaptable to be moved automatically or semi-automatically. In some embodiments of the device, the tissue collection element comprises stainless steel. In some embodiments of the tissue collection element, a portion of the tissue collection element comprises a shape memory alloy. Additionally, at least a portion of the tissue collection element can be coated with a non-friction coating, such as Teflon®, poly(tetrafluoroethylene), perfluoroalkoxy polymer resin, fluorinated ethylene-propylene, fluoropolymers, and combinations thereof. The distal end of the outer needle can be coated with a non-friction coating. In some embodiments, the distal end of the tissue collection element comprises a beveled cutting edge. The tissue collection element can be disposable. The tissue collection element can also cut and receive tissue within the tissue collection element without further damaging the tissue. Additionally, the device can further comprise a stylet, wherein the stylet is adaptable to be inserted into the tissue collection element. A negative pressure source adaptable to facilitate application of negative pressure to the distal end of the tissue collection element can also be used with the device. The negative pressure can be supplied by a syringe, such as a two-stage or multi-stage syringe. The outer needle can be echogenic. Alternatively, the tissue collection element can be echogenic. In some embodiments, both the outer needle and the tissue collection element can be echogenic. The echogenicity of the outer needle and the tissue collection element can be facilitated by rotational actuation applied to the outer needle or the tissue collection element. Alternatively, the echogenicity of the outer needle and the tissue collection element can be facilitated by vibrations induced at the distal tip of the outer needle. In some embodiments of the biopsy device, the biopsy device further comprises a cannula wherein the cannula is adaptable to contain the tissue collection element and outer needle and wherein the cannula is further translatable and rotatable relative to the tissue collection element and the outer needle. Additionally, the biopsy device can further comprise a depth gauge adaptable to assess the depth of penetration of the tissue collection element within a target location. A depth stop also can be included with the device, the depth stop adaptable to be set to limit the depth of penetration of the tissue collection element within a target location. The biopsy device described herein comprises a tissue collection element adaptable to capture a measurable target tissue sample from a collection region in at most three passes. One or more radiopaque markers on at least a portion of the length of the device can be included with the device. The device can be adaptable to be operated using single-hand operation. In some embodiments, the device can further comprise a quick excursion element adaptable to repeatedly extrude depth limited portions of target tissue. The outer needle can be a needle with a gauge between 18 and 27. The distal end of the tissue collection element can extract a portion of target tissue from a collection region having a diameter greater than 0.05 inches in diameter. Additionally, the device can further comprise an endoscope, wherein the position of the biopsy device can be adjusted to accommodate the working length of the endoscope.

Further provided herein is a biopsy device comprising: a stylet having a proximal end and a distal end wherein the stylet is adaptable to be inserted inside the tissue collection element; and a tissue collection element formed from material having a first constrained configuration when the stylet is inserted inside said tissue collection element and a second unconstrained configuration when the stylet is retracted from within the tissue collection element, wherein the tissue collection element is translationally and rotationally moveable in response to actuation by the drive mechanism. The distal tip of the tissue collection element deviates from the axis of rotation of said tissue collection element from 0 degrees to 180 degrees and rotates around said axis of rotation from 0 degrees to about 360 degrees. Furthermore, the distal end of the tissue collection element deviates from the axis of rotation of said tissue collection element along an angle, radius, helical path, or contour. The distal end of the tissue collection element can comprise an opening wherein the opening obtains a portion of tissue having a cross-sectional diameter greater than the cross-sectional diameter of the tissue collection element in its constrained configuration. In some embodiments, the tissue collection element can be translated to a target location during tissue acquisition. The distal end of the tissue collection element can be rotationally actuated to produce a rotational motion. The rotational motion is a motion selected from the group consisting of continuous, intermittent, reciprocating, and combinations thereof. The tissue collection element can be adaptable to be moved manually. Alternatively, the tissue collection element can be adaptable to be moved automatically or semi-automatically. In some embodiments, the tissue collection element comprises stainless steel. In some embodiments, at least a portion of the tissue collection element comprises a shape memory alloy. The distal end of the tissue collection element can be coated with a non-friction coating. The distal end of the tissue collection element can comprise a beveled cutting edge. In some embodiments, the tissue collection element is disposable. The tissue collection element can cut and receive tissue within the tissue collection element without further damaging the tissue. In some embodiments, the stylet can be adaptable to penetrate tissue as the tissue collection element is advanced toward the target location. Additionally, the stylet can be adaptable to preclude anomalous tissue acquisition as the tissue collection element is advanced toward the target location. The stylet can be adaptable to expel a biopsy tissue sample from the tissue collection element. In some embodiments, the device can further comprise a negative pressure source adaptable to facilitate application of negative pressure to the distal tip of the tissue collection element. The negative pressure can be supplied by a syringe, for example purposes only, a two-stage syringe or multi-stage syringe. In some embodiments, the tissue collection element is adaptable to be echogenic. The echogenecity of the tissue collection element can be facilitated by rotational actuation applied to the tissue collection element. Alternatively, the echogenecity of the tissue collection element can be facilitated by vibrations induced at the distal end of the tissue collection element. In some embodiments, the device can Anther comprise a cannula adaptable to contain the tissue collection element, the cannula further translatable and rotatable relative to the tissue collection element. In some embodiments, the device can further comprise a depth gauge adaptable to assess the depth of penetration of the tissue collection element within a target location. Additionally, the device can comprise a depth stop adaptable to set a limit for the depth of penetration of the tissue collection element within a target location. The tissue collection element can be adaptable to capture a measurable target tissue sample from a collection region in at most three passes. In some embodiments, the device can further comprise one or more radiopaque markers on at least a portion of the length of the device. The device can be adaptable to be operated using single-hand operation. In some embodiments, the device can further comprise a quick excursion element adaptable to repeatedly extrude depth-limited portions of target tissue. In some embodiments, the tissue collection element further comprises a shaft located between the proximal end and the distal end, the shaft having comprising a gauge of 18 to 27. The distal tip of the tissue collection element is adaptable to extract a portion of target tissue from a collection region having a diameter greater than 0.05 inches in diameter. Additionally, the device can further comprise an endoscope, wherein the position of the biopsy device can be adjusted to accommodate the working length of the endoscope.

Further provided herein is a biopsy device comprising: an outer needle having a proximal end and a distal end wherein the proximal end is connectable to a drive mechanism; and a tissue collection element comprising a proximal end, a distal end, and a helical cutting edge along at least a portion of the length of the tissue collection element, wherein the helical cutting edge is adaptable to cut a portion of tissue from a target tissue; and a non-friction coating adaptable to be applied to at least a portion of the distal end of the tissue collection element, wherein the tissue collection element is translationally and rotationally moveable within the outer needle and distally beyond the distal end of the outer needle in response to actuation by the drive mechanism. Additionally, a non-friction coating can be applied to at least a portion of the outer needle. The helical cutting edge of the device can extend radially from a solid core. Alternatively, the helical cutting edge can be adaptable to encircle a hollow core. The tissue collection element is translated from within the outer needle to a target location. In some embodiments, the distal end of the tissue collection element is rotationally actuated to produce a rotational motion. The rotational motion can be a motion selected from the group consisting of continuous, intermittent, reciprocating, and combinations thereof. In some embodiments, the tissue collection element is adaptable to be moved manually. Alternatively, the tissue collection element can be adaptable to be moved automatically or semi-automatically. In some embodiments, the tissue collection element comprises stainless steel. Furthermore, at least a portion of the distal end of the tissue collection element comprises a beveled cutting edge. The tissue collection element can be disposable. The tissue collection element can cut and receive tissue within the outer needle without further damaging the tissue. In some embodiments, the device further comprises a stylet adaptable to be inserted in at least one of the outer needle and the tissue collection element. In some embodiments, the device can further comprise a negative pressure source adaptable to facilitate application of negative pressure to the distal end of at least one of the outer needle or the tissue collection element. The negative pressure can be supplied by a syringe, for example purposes only, a two-stage syringe or a multi-stage syringe. The outer needle can be echogenic. Alternatively, the tissue collection element can be echogenic. In some embodiments, both the outer needle and the tissue collection element can be echogenic. The echogenicity of the outer needle and the tissue collection element can be facilitated by rotational actuation applied to the outer needle or the tissue collection element. Alternatively, the echogenicity of the outer needle and the tissue collection element can be facilitated by vibrations induced at the distal tip of the outer needle. In some embodiments of the biopsy device, the biopsy device further comprises a cannula wherein the cannula is adaptable to contain the tissue collection element and outer needle and wherein the cannula is further translatable and rotatable relative to the tissue collection element and the outer needle. Additionally, the biopsy device can further comprise a depth gauge adaptable to assess the depth of penetration of the tissue collection element within a target location. A depth stop also can be included with the device, the depth stop adaptable to be set to limit the depth of penetration of the tissue collection element within a target location. The biopsy device described herein comprises a tissue collection element adaptable to capture a measurable target tissue sample from a collection region in at most three passes. Additionally, the device can further comprise an endoscope, wherein the position of the biopsy device can be adjusted to accommodate the working length of the endoscope.

Further provided herein is a biopsy device comprising an outer needle having a proximal end and a distal end wherein the proximal end is connected to a drive mechanism; a tissue collection element formed from material having a first constrained configuration when positioned within the cannula prior to deployment and a second unconstrained configuration when extended distally beyond the distal end of the cannula wherein the tissue collection element is translationally and rotationally moveable within the cannula and distally beyond the distal end of the cannula in response to actuation by the drive mechanism, wherein the distal end of said tissue collection element forms an opening adaptable to obtain a target tissue from a collection region, the opening having a cross-sectional diameter greater than the cross-sectional diameter of the outer needle.

Further provided herein is a biopsy device comprising a stylet having a proximal end and a distal end wherein the stylet is adaptable to be inserted inside the tissue collection element; and a tissue collection element having a proximal end and a distal end, the tissue collection element formed from material having a first constrained configuration when the stylet is inserted inside said tissue collection element and a second unconstrained configuration when the stylet is retracted from within the tissue collection element wherein the tissue collection element is translationally and rotationally moveable in response to actuation by the drive mechanism, wherein the distal end of said tissue collection element forms an opening adaptable to obtain a target tissue from a collection region, the opening having a cross-sectional diameter greater than the cross-sectional diameter of the outer cannula.

In some embodiments, provided herein, is a biopsy device comprising an outer needle having a proximal end and a distal end wherein the proximal end is connected to a drive mechanism; and a tissue collection element formed from material having a first constrained configuration when positioned within the outer needle prior to deployment and a second unconstrained configuration when extended distally beyond the distal end of the outer needle wherein the tissue collection element is translationally and rotationally moveable within the outer needle and distally beyond the distal end of the outer needle in response to actuation by the drive mechanism; and a non-friction coating. The non-friction coating can be applied to the distal end of the outer needle. Additionally, the non-friction coating can be applied to the distal end of the tissue collection element.

Further provided herein is a biopsy device comprising a stylet having a proximal end and a distal end wherein the stylet is insertable inside the tissue collection element; a tissue collection element formed from material having a first constrained configuration when the stylet is inserted inside said tissue collection element and a second unconstrained configuration when the stylet is retracted from within the tissue collection element wherein the tissue collection element is translationally and rotationally moveable in response to actuation by the drive mechanism; and a non-friction coating applied to some portion of the distal tip of the tissue collection element.

Also provided herein is a method for obtaining a measurable target tissue from a collection region comprising: inserting a biopsy device comprising an outer needle having a proximal end and a distal end wherein the proximal end is connected to a drive mechanism, and a tissue collection element formed from material having a first constrained configuration when positioned within the outer needle prior to deployment and a second unconstrained configuration when extended distally beyond the distal end of the outer needle wherein the tissue collection element is translationally and rotationally moveable within the outer needle and distally beyond the distal end of the outer needle in response to actuation by the drive mechanism; advancing the tissue collection element into a patient toward a target tissue; excising a measurable amount of target tissue with the tissue collection element; and removing the excised target tissue from the patient. Additionally, the method can further comprise the step of transmitting a translational actuation force to at least one of the outer needle and the tissue collection element. In some embodiments, the method can further comprise the step of transmitting a rotational actuation force to the tissue collection element. The excising step can further comprises procuring a tissue sample by rotating the tissue collection element while translating the outer needle and tissue collection element. In some embodiments, the method can further comprise the step of step of inserting a stylet into the tissue collection element. Furthermore, the method can further comprising the step of applying negative pressure to the distal tip of at least one of the tissue collection element and cannula. The step of approaching the target location with the stylet inserted in the tissue collection element prior to sample acquisition.

In some embodiments, a method for obtaining a measurable target tissue from a collection region is provided herein, the method comprising: inserting a biopsy device comprising a stylet having a proximal end and a distal end wherein the stylet is insertable inside the tissue collection element; and a tissue collection element formed from material having a first constrained configuration when the stylet is inserted inside said tissue collection element and a second unconstrained configuration when the stylet is retracted from within the tissue collection element wherein the tissue collection element is translationally and rotationally moveable in response to actuation by the drive mechanism; advancing the tissue collection element into a patient toward a target tissue; excising a measurable amount of target tissue with the tissue collection element; and removing the excised target tissue from the patient. In some embodiments, the method can further comprise the step of transmitting a translational actuation force to the tissue collection element. Alternatively, the method can comprise the step of transmitting a rotational actuation force to the tissue collection element. In some embodiments of the method, the excising step further comprises procuring a tissue sample by rotating the tissue collection element while translating the tissue collection element. The excised tissue can further be removed from the biopsy device. The stylet can be used to remove the excised tissue. In some embodiments, the method can include the application of negative pressure to the distal tip of the tissue collection element. The stylet in the tissue collection element can also be used to approach the target location prior to sample acquisition.

Another method provided herein, is a method for obtaining a target tissue from a collection region comprising: inserting a biopsy device comprising a cannula having a proximal end and a distal end wherein the proximal end is connectable to a drive mechanism; and a tissue collection element having helical cutting features along a portion of its length at the distal end thereof wherein the tissue collection element is adapted to cut target tissue from a collection region and is translationally and rotationally moveable within the outer needle and distally beyond the distal end of the outer needle in response to actuation by the drive mechanism; and a non-friction coating applied to some portion of the distal tip of the tissue collection element and/or the outer needle; advancing the tissue collection element into a patient toward a target tissue; excising a measurable amount of target tissue with the tissue collection element; and removing the target tissue from the patient. In some embodiments, the method can further comprise the step of transmitting a translational actuation force to the cannula and/or tissue collection element. Alternatively, the method can further comprise the step of transmitting a rotational actuation force to the tissue collection element. Furthermore, the excising step further comprises procuring a tissue sample by rotating the tissue collection element while translating the outer needle and tissue collection element. In some embodiments, the negative pressure can be applied to the distal end of at least one of the tissue collection element or outer needle prior to sample acquisition. The method can also provide for the step of approaching the target location with the stylet inserted in the outer needle or tissue collection element prior to sample acquisition.

Further provided herein is a kit for obtaining a measurable target tissue from a collection region comprising: a removable handle containing a drive mechanism; one or more cannula outer needles, each cannula outer needle having a proximal end and a distal end wherein the proximal end is adaptable to engages the drive mechanism; and one or more tissue collection elements, each tissue collection element having an adapted and configured form to receive a measurable target tissue from a collection region wherein the tissue collection element is translationally and rotationally moveable within the outer needle cannula distally beyond the distal end of the outer needle cannula in response to the drive mechanism.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 is an exploded view of one embodiment of a biopsy device;

FIG. 2 is an illustration of a lateral cross-section of one embodiment of an actuation module used with the device;

FIG. 3 depicts one embodiment of a negative pressure module used with the device;

FIG. 4 is an illustration of a lateral cross-section of a depth penetration module used with the device;

FIG. 5 is an illustration of a device being assembled as viewed from the side;

FIG. 6 is a perspective view of one embodiment of an assembled device;

FIG. 7 is a static illustration of the device of FIG. 6 in use;

FIG. 8 is an exploded view on another embodiment of a biopsy device;

FIG. 9A is an illustration of an alternate embodiment of a actuation module; FIG. 9B is a perspective view of the actuation module; FIG. 9C is another perspective view of the actuation module; FIG. 9D is a cross-sectional view of the actuation module;

FIG. 10A is a cross-sectional view of one embodiment of a gear motor of a rotation actuation module as viewed from the side; FIG. 10B is a view of the gear motor as viewed from the side;

FIG. 11 depicts an isolated view of another embodiment of a negative pressure device;

FIGS. 12A-12D is an illustration of the steps to prepare the device for operation;

FIG. 13A is a lateral cross-sectional view of one embodiment of a catheter module; FIG. 13B is a cross-section of the catheter module of FIG. 13A along the line B-B; FIG. 13C is an isolated view of a tissue collection element in a first configuration; FIG. 13D is an isolated view of a tissue collection element in a second configuration; FIG. 13E is a side view of a catheter module; FIG. 13F is a cross-section of FIG. 13E along the line F-F; FIG. 13G is a cross-section of FIG. 13E along the line G-G.

FIG. 14A is a side view of a catheter module comprising a bent tube tissue collection element; FIG. 14B is a cross-sectional view of FIG. 14A along the line B-B; FIG. 14C is a cross-sectional view of FIG. 14A along the line C-C.

FIG. 15A depicts one embodiment of a drill-bit tissue collection element for use with a biopsy device; FIG. 15B is a lateral cross-sectional view of a catheter module comprising a drill-bit tissue collection element; FIG. 15C is a side view of a drill-bit catheter module; FIG. 15D is a cross-sectional view of FIG. 15C along the line D-D; FIG. 15E is a cross-sectional view of FIG. 15C along the line D-D;

FIG. 16A is another embodiment of a drill-bit tissue collection element; FIG. 16B is a lateral cross-sectional view of the catheter module comprising a drill-bit tissue collection element; FIG. 16C is a side view of a drill-bit catheter module; FIG. 16D is a cross-sectional view of FIG. 16C along the line D-D;

FIG. 17A is an embodiment of a bent tube tissue collection element with a low-friction coating; FIG. 17B is a view of the device of FIG. 17A as viewed from the end;

FIGS. 18A-18D illustrate a biopsy device in use; and

FIG. 19 is an illustration of alternative embodiment of the biopsy device in use.

### DETAILED DESCRIPTION OF THE INVENTION

The biopsy devices described herein can be designed to automate the procedure for the diagnosis of suspect areas of tissue. The device can be used with tumors and cysts, or any other suitable soft tissue from which a sample can be obtained. Various embodiments of the device are provided herein. In one embodiment, the device described herein can comprise an outer needle having a distal end, or end closer to the body, and a proximal end, or end closer to the exterior of the body, connected to a drive mechanism and a tissue collection element having a distal end and a proximal end. The distal end of the tissue collection element can be formed from a material having a first constrained configuration when positioned within the outer needle prior to deployment and a second unconstrained configuration when extended distally beyond the distal end of the outer needle, wherein the tissue collection element is translationally and rotationally moveable within the outer needle and distally beyond the distal end of the outer needle in response to actuation by the drive mechanism. The outer needle can have a gauge of 18 to 27.

In some embodiments, the tissue collection element has a tube structure. The tissue collection element can be formed such that it has a first configuration that is constrained when positioned, for example, within an outer needle, and a second configuration when extended distally beyond the distal end of the outer needle. Once extended, and unconstrained, the tissue collection element biases away from a central axis such that the tubular structure of the tissue collection element bends, forming a bent tube. When introduced proximally to the tissue to be sampled, the tissue collection element can be housed within the outer needle. The outer needle holds the tissue collection element in a first configuration which follows the structure of the outer needle. When a tissue sample is to be extracted, the distal end of tissue collection element can be extended past the distal end of the outer needle. Once the distal end of the tissue collection element extends past the distal end of the outer needle, the tissue collection element can change configuration to a second configuration. In the second configuration, the tissue collection element biases away from the central axis of the outer needle. In some embodiments, the tissue collection element deviates from the central axis from about 0 to about 180 degrees. This enlarges the cross-section of the opening in the distal end of the tissue collection element. The radial deviation of the distal end of the tissue collection element from the rotational axis is greater than the radius of the outer needle. This enables a larger amount of tissue to be excised by the tissue collection element. The tissue collection element can be made of stainless steel. Alternatively, the tissue collection element, or solely a portion of the distal tip thereof, can be made of a shape memory material, for example, Nitinol. The tissue collection element can be made of any suitable material that can exist in at least two configurations. In some embodiments, the distal end of the tissue collection element comprises a beveled edge to facilitate the cutting of tissue. The enlarged opening of the tissue collection element, together with the rotational motion of the tissue collection element enables the tissue collection element to capture a larger amount of tissue in a single pass, as compared to conventional methods. A sufficient amount of sample can be obtained from the tissue of interest in approximately three passes. In some embodiments, a sufficient amount of sample can be obtained in a single pass.

The tissue collection element can transition between two configurations during use. As previously mentioned, the tissue collection element can exist in a first configuration before the sample is to be obtained. The tissue collection element is constrained in a first configuration by an external structure. When in use, the tissue collection element is no longer constrained by a constraining structure and transitions to a second configuration. In some embodiments, the constraining structure is an outer needle. The outer needle constrains the tissue collection element in a first configuration. In some embodiments, the constraining structure is a stylet located in tissue collection element. The stylet can constrain the tissue collection element in a first configuration. Once the stylet is retracted from the proximal end of the tissue collection element, the tissue collection element can transition to a second configuration.

In some embodiments, the stylet can be used to facilitate the penetration of the device to position the tissue collection element in proximity to the tissue of interest. The stylet additionally can preclude the capturing of anomalous tissue by the tissue collection element, as the tissue collection element is advanced toward the tissue to be sampled. Once the tissue collection element is in position, proximal to the tissue of interest, the stylet can be retracted and sample excised and collected by the tissue collection element. The stylet can further be used, in some embodiments, to remove the tissue collected in the tissue collection element.

In another embodiment, the tissue collection element comprises a rotatable cutting or boring tool having two or more helical cutting edges. The revolving tissue collection element can be adapted to provide flats or flutes for the capture and release of cut tissue. The rotatable cutting or boring tissue collection element has a drill-bit-like configuration. The drill-bit like structure comprises a helical cutting edge located on the exterior of the tissue collection element. In some embodiments, the helical cutting edge extends radially from a center core of the tissue collection element. In some embodiments, the helical cutting edge wraps around a center portion of the tissue collection element; the center portion of the tissue collection element remains hollow.

The distal end of the tissue collection element can then rotate around the central axis of the outer needle. The tissue collection element can rotate about the center axis from about 0 degrees to about 360 degrees. The distal end of the tissue collection element can be actuated to cause rotational motion of the distal tip of the tissue collection element. The motion can be any suitable rotational motion including, but not limited to, continuous motion, intermittent motion, and reciprocating motion, and any combination thereof. In some embodiments, the rotation of the tissue collection element is controlled by the drive mechanism in the actuation module housing the drive mechanism. In addition to rotational actuation, the tissue collection element can be linearly translated. The rotational actuation and linear translation of the tissue rotation element can be affected by the user. In such an embodiment, the tissue collection element is said to be manually operated. Alternatively, the linear translation can be affected manually, while the rotational actuation can be affected by the drive mechanism. In such an embodiment, the tissue collection element is semi-automatically operated. In some embodiments, both the linear translation and the rotational motion of the tissue collection element are affected by the drive mechanism. In such an embodiment, the movement of the tissue collection element is considered to be automatic.

A feature of the invention provided herein is the ease of which the tissue collection element can be visualized during a procedure. The visualization of the tissue collection element can be done using the echogenicity of the tissue collection element. Small motion of the tissue collection element can enhance the echogenicity of the tissue collection element. The echogenicity of the tissue collection element can be enhanced or facilitated by rotation of the tissue collection element. The echogenicity of the tissue collection element can also be enhanced or facilitated by vibrations induced at the distal tip of the tissue collection element. The vibrations can be actuated by a piezoelectric element. The vibrations can be actuated by any suitable vibration source. In some embodiments, the outer needle has enhanced echogenicity. The echogenicity of the tissue collection element can be enhanced or facilitated by rotation of the tissue collection element. The echogenicity of the tissue collection element can also be enhanced or facilitated by vibrations induced at the distal tip of the outer needle. In some embodiments, both the tissue collection element and the outer needle have enhanced echogenicity. In some embodiments, the tissue collection element can be visualized by using radiopaque markers located along at least a portion of the tissue collection element. In some embodiments, radiopaque markers are located along at least a portion of the outer needle.

In some embodiments, the tissue collection element is disposable while the handle can be reusable. In some embodiments, the entire device is disposable. Alternatively, in some embodiments, the entire device can be reusable.

The device can be designed so that the device can be operated by single-handed operation. In some embodiments, the distal end of the tissue collection element can be coated with a non-friction coating. Current embodiments of biopsy devices are not coated with a non-friction coating. Current biopsy devices poke a sample in a single direction with a tissue collection element. These devices require friction to capture a tissue sample, since friction is used to capture the sample inside the tissue collection element. The invention disclosed herein does not require the use of friction due to the physical structure and rotation of the tissue collection element. The tissue collection element disclosed herein can be coated with a non-friction coating. In some embodiments, the entire distal end of the tissue collection element is coated. In some embodiments, the inside of the tissue collection element is coated with a non-friction coating. The non-friction coating can facilitate the translation and rotation of the tissue collection element through the tissue sample. Examples of non-friction coatings include, but are not limited to poly(tetrafluoroethylene), perfluoroalkoxy polymer resin, fluorinated ethylene-propylene, fluoropolymers, combinations thereof, or any other suitable non-friction coating.

### I. Devices

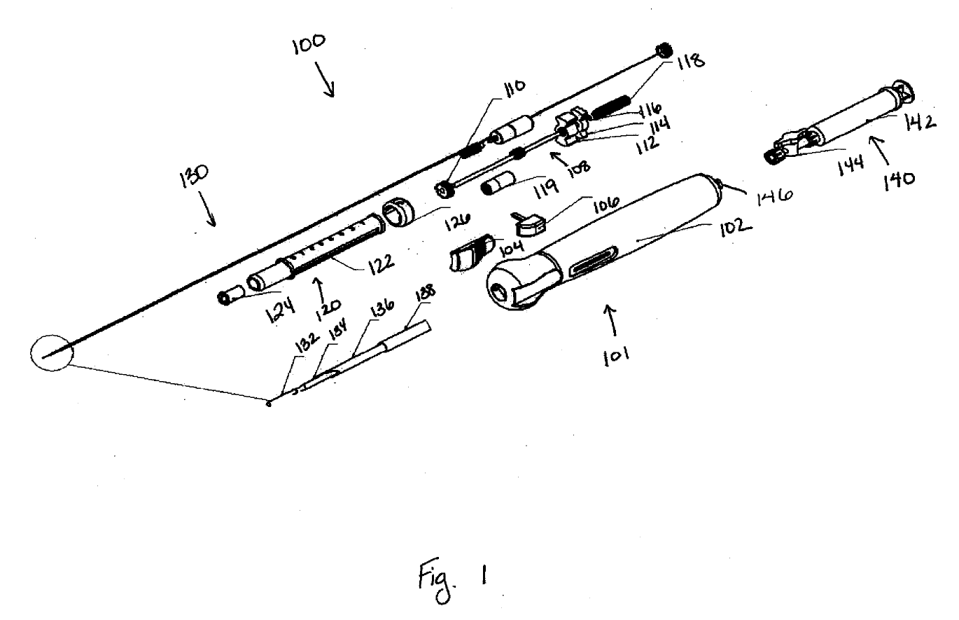

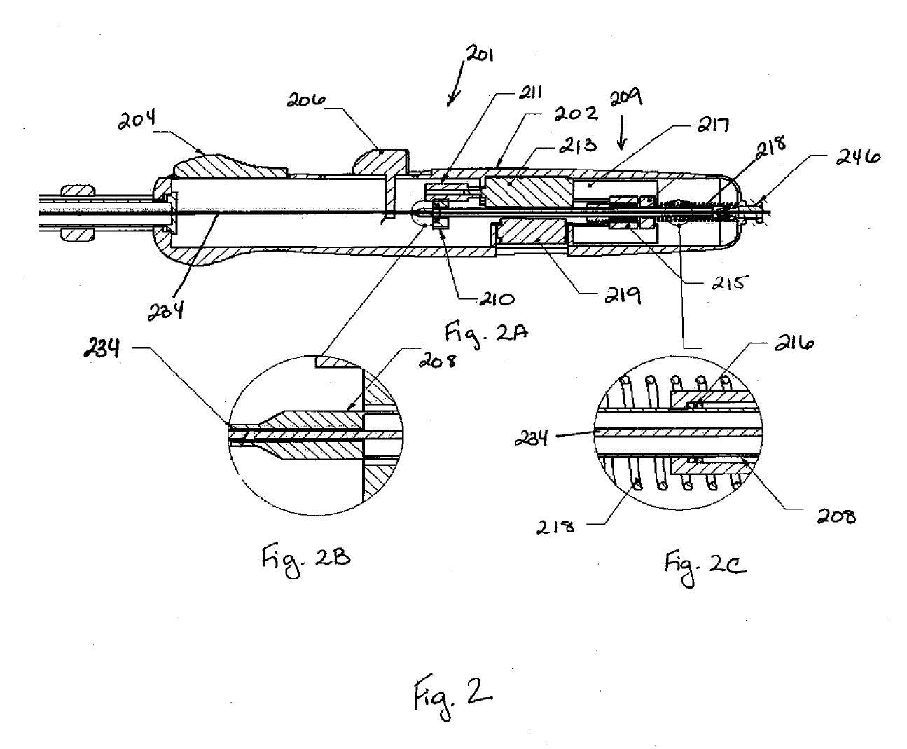

FIG. 1 illustrates an exploded view of one embodiment of a biopsy device 100. The biopsy device 100 can be comprised of both reusable and disposable parts. As shown in FIG. 1 the device 100 comprises an actuation module 101 comprising a handle 102 which can comprise a motor control switch 104 and a quick excursion switch 106, and a negative pressure barrel 108 inside the handle comprising at least one gear 110, at least one internal threaded slider 112, a washer 114, an o-ring 116, and a spring 118. The handle 102 of the device is in mechanical communication with a depth penetration module 120. The depth penetration module 120 can further comprise a tissue collection element depth gauge 122 and a luer adaptor 124 for endoscopic assembly. The tissue element depth gauge 122 can additionally include a tissue collection element depth stop 126. The device 100 further can farther comprise a catheter module 130. The catheter module 130 can comprise a tissue collection element 134 and an outer cannula 138. In some embodiments, the catheter module 130 further comprises a stylet 132, as shown in FIG. 1. Additionally, the catheter module 130 can comprise an outer needle 136. FIG. 1 illustrates a catheter module 130 comprising a stylet 132 nested within a tissue collection element 134 which is nested within an outer needle 136. The outer needle 136 can then be nested in the cannula 138, as shown in FIG. 1. In some embodiments of the device 100, an external negative pressure module 140 can be in communication with the actuation module 101, as shown in FIG. 1. The negative pressure module 140 can comprise a syringe 142 and a luer adaptor 144. The luer adaptor 144 on the negative pressure module 140 together with the luer adaptor 146 on the actuation module 101 provides communication between the negative pressure module 140 and the actuation module 101. In some embodiments, the actuation module is powered by a battery 119.

FIG. 2A is a lateral cross-sectional view of the actuation module 201. The actuation module 201 comprises a handle 202, a motor control switch 204, and a quick excursion switch 206 on the exterior of the actuation module 201. Additionally, the actuation module can comprise a luer adaptor 246. The luer adaptor 246 can be used to connect a negative pressure module to the exterior of the of the actuation module 201. In some embodiments of the biopsy device, a stylet can be connected to actuation module 201 through the luer adaptor 246. Any suitable component can be connected to the exterior of the actuation module 201. As shown in FIG. 2B, in the interior of the actuation module 201, the proximal end of the tissue collection element 234 can be rigidly connected to the negative pressure barrel 208. Negative pressure can be applied to the area from which tissue is being excised through the tissue collection element 234.

The actuation module further comprises a drive mechanism 209. The drive mechanism 209 comprises an assembly for actuating the tissue. In some embodiments, the drive mechanism causes rotational actuation of the tissue collection element. In some embodiments, the drive mechanism causes translational actuation of the tissue collection element. The drive mechanism can cause both rotational and translational actuation of the tissue collection element. The proximal end of the tissue collection element 234 is attached to a gear 210 in the actuation module 201. The movement of the gear 210 can facilitate rotation of the tissue collection element 234. The gear 210 attached to the tissue collection element is in communication with a second gear 211. In some embodiments, the communication is mechanical communication. The second gear 211 is in communication with a gear motor 213. In some embodiments, the communication between the second gear 211 and the gear motor 213 is mechanical communication. The gear of the actuation module can be driven by a motor, a spring, gear assembly, or any other suitable mechanism for rotating the gear. The rotation of the gears 210, 211 causes the distal end of the tissue collection element 234 to rotate. The gear motor can be manually powered, for example, using a wind-up mechanism Alternatively, the gear motor can be electrically powered. The gear motor can be powered by a battery 219, as shown in FIG. 2A. The battery can be a rechargeable battery or can be a replaceable battery. In some embodiments, an external power source can be used to power the gear motor.

In some embodiments, the actuation module translates the tissue collection element linearly in addition to rotating the tissue collection element. The actuation module 201 can further comprise an internal threaded slider 215. The slider 215 can facilitate linear translation of the tissue collection element 234. Guides 21 7 are further present that restrict the slider from rotating with the tissue collection element. A spring 218 in communication with the slider 215 can facilitate translation of the slider 215. FIG. 2C illustrates the negative pressure barrel 208 in communication with a luer adaptor 246. At least one o-ring 216 can be used to facilitate forming a seal to create negative pressure inside the negative pressure barrel 208.

In some embodiments, the actuation module comprises a quick excursion switch 206, as shown in FIG. 2. The quick excursion switch 206 can be connected to the tissue connection element 234 and/or the negative pressure barrel 208. In some embodiments, the quick excursion switch is rigidly connected to the tissue collection element 234 and/or the negative pressure barrel 208.

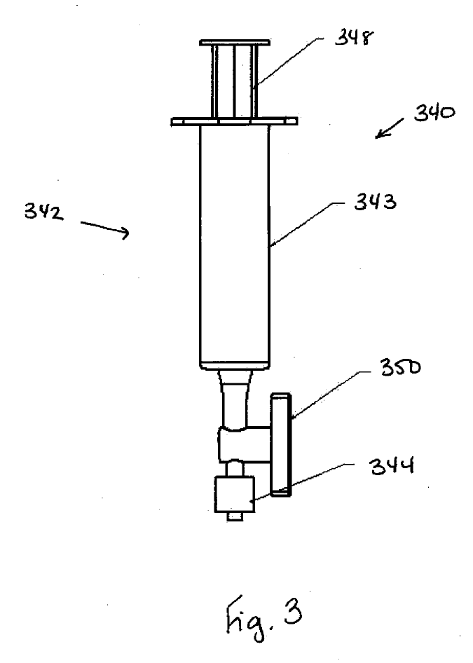

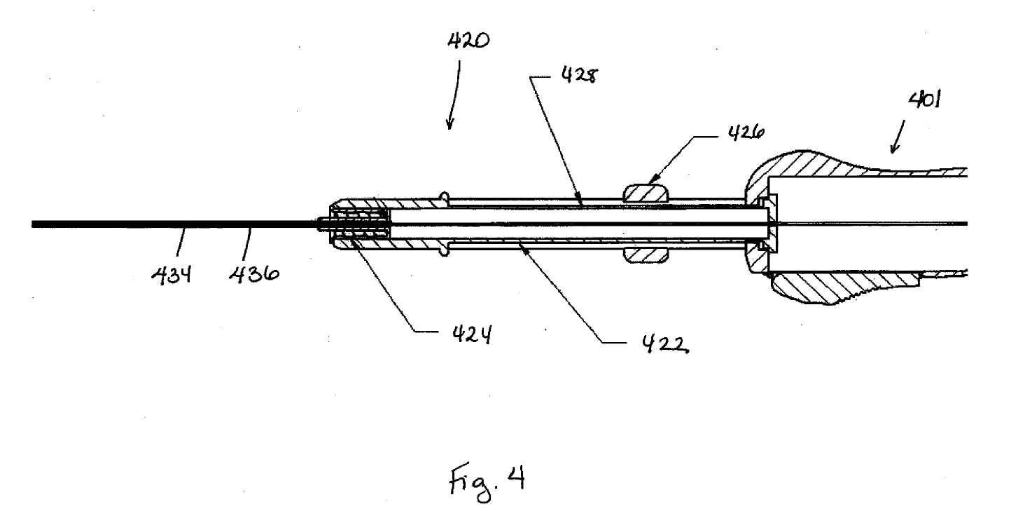

FIG. 3 is a side view of one embodiment of a negative pressure module 340 that can be used with the biopsy device. In some embodiment, the negative pressure module is a syringe 342. The negative pressure module 340 can comprise a barrel 343, a plunger 348 able to reciprocate within the barrel 343, and a valve 350. The negative pressure module 340 can be used to create negative pressure. Negative pressure created can be applied through the tissue collection element to the area from which tissue is to be excised. The negative pressure module 340 can be connected to the actuation module through the luer adaptor 344. The valve 350 of the negative pressure module 340 has an open position and a closed position. The valve can be in either the open or closed position when connected to the actuation module. Once the negative pressure module 340 has been connected to the device, the valve 350 is positioned in the closed position if not already in the closed position. The plunger 348 of the syringe 342 can then be drawn back to create negative pressure inside the barrel 343. Once the tissue collection element has been actuated, the valve 344 can be turned to the open position so that the negative pressure inside the negative pressure module passes through to the negative pressure barrel inside the actuation module. The negative pressure in the negative pressure barrel creates negative pressure inside the tissue collection element, thereby facilitating drawing the tissue sample into the tissue collection element. As will be appreciated by those skilled in the art, any suitable method or force for drawing tissue into the collection element can be used.

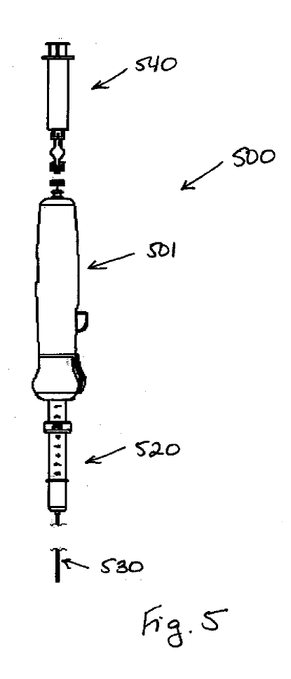

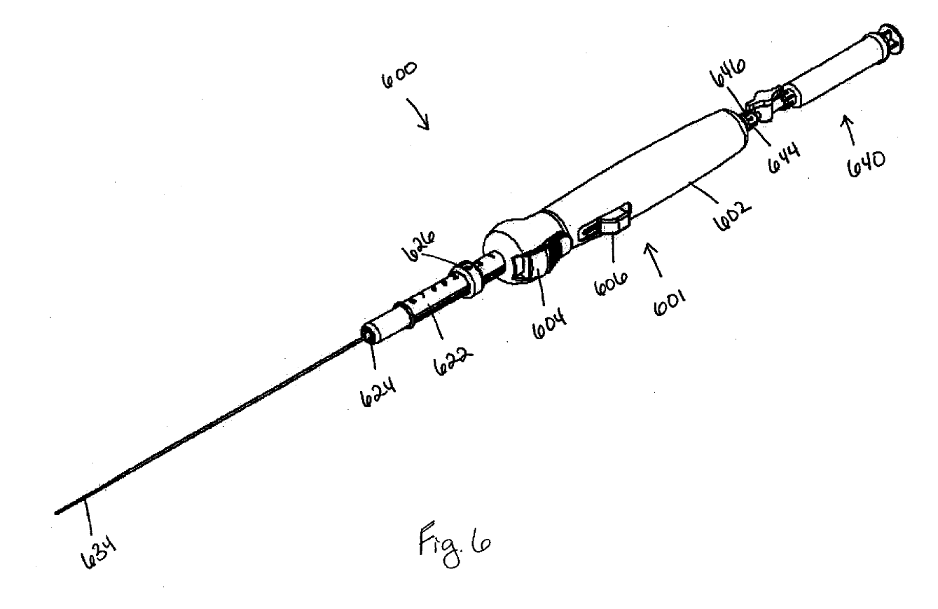

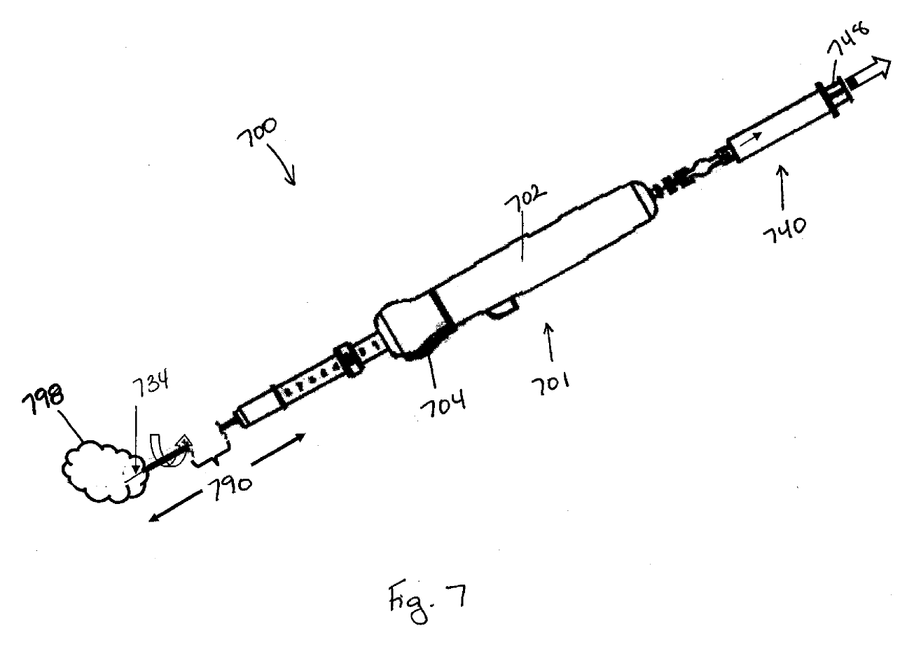

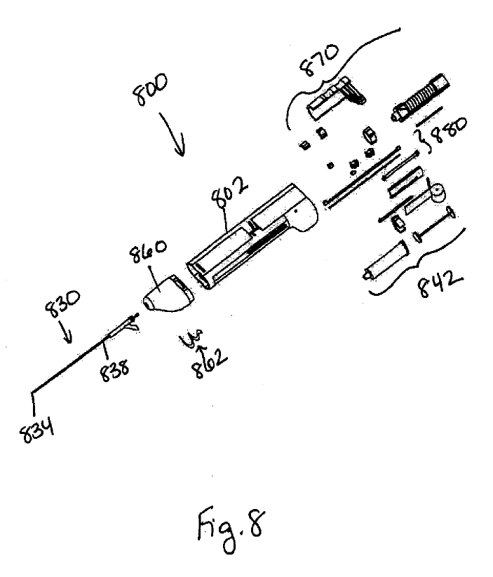

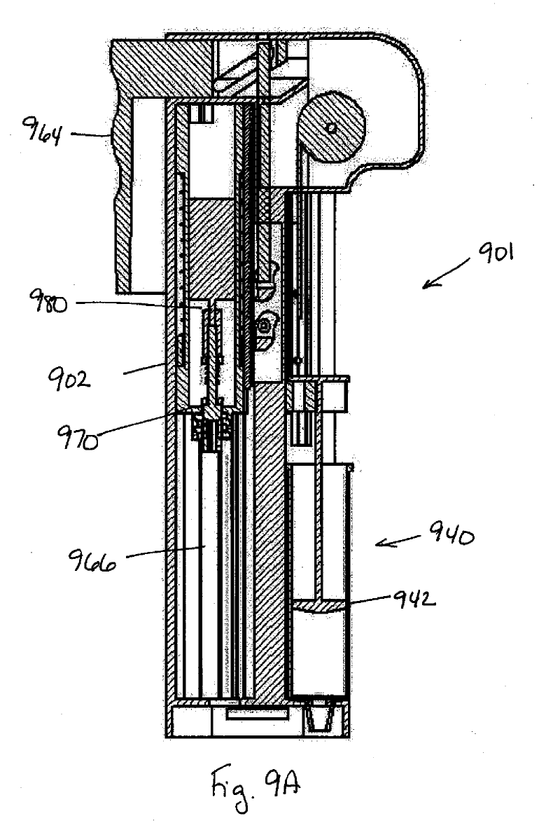

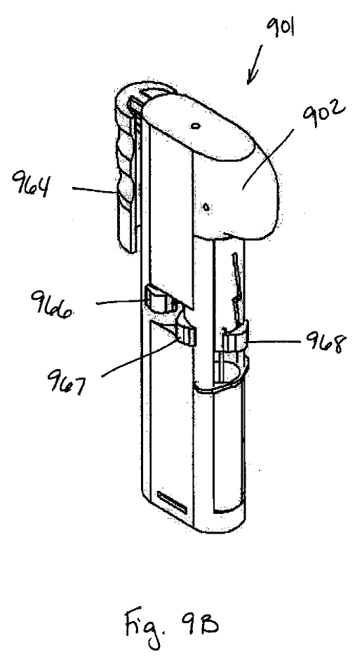

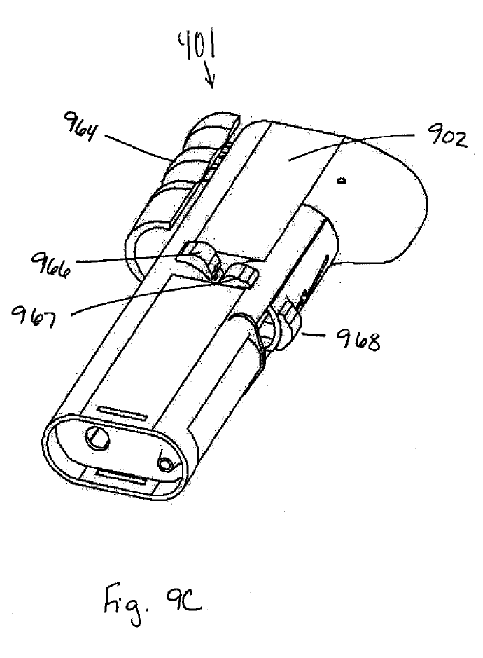

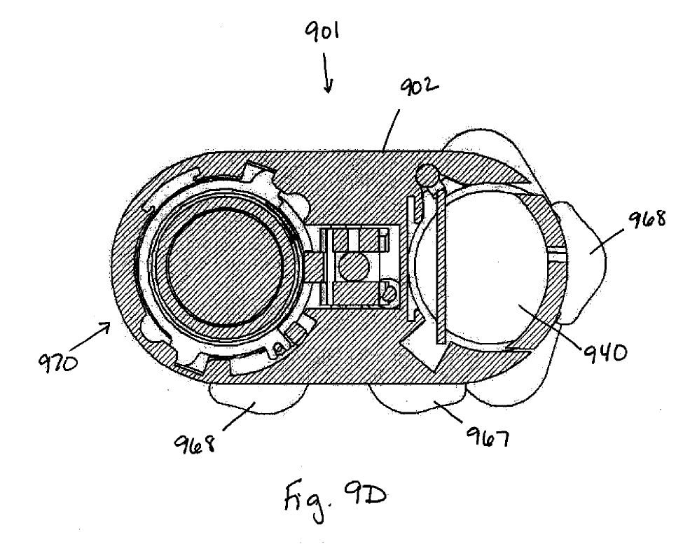

FIG. 4 is a lateral cross-section through one embodiment of a depth penetration module 420. The depth penetration module 420 is slidably connected to the distal end of the actuation module. The depth penetration module 420 can comprise a depth gauge 422, with a depth stop 426, and a luer adaptor 424. The depth penetration module 420 can fiber comprise a translation guide 428 for automated translation of the tissue collection element in a distal and proximal direction. The tissue collection element 434 passes through the depth penetration module 420. The outer needle 436 is connected to the tissue collection element to the depth penetration module 420 at the luer adaptor 424. The depth gauge 422 controls the depth to which the tissue collection element can be inserted into the tissue sample. The depth can be set using the depth stop 426. In some embodiments, the depth to which the tissue collection element passes into the tissue can be automatically controlled by the biopsy device. In some embodiments, the depth to which the tissue collection element passes into the tissue can be controlled manually, while using the depth gauge to determine the extent to which the tissue collection element has been inserted into the tissue.

FIG. 5 is a side view of a biopsy device 500 as the device is being assembled. The proximal end of the catheter module 530 is connected to the distal end of the depth penetration module 520. The proximal end of the depth penetration module is slidably connected to the distal end of the actuation module 501. In some embodiments, a negative pressure module 540 is connected to the proximal end of the actuation module, as is shown in FIG. 5.

FIG. 6 is an illustration of a perspective view of the device 600 as assembled. As shown in FIG. 6, the negative pressure module 640 is located on the distal end of the actuation module 601 and is in mechanical communication with the actuation module 601 through luer adaptors 644, 646. As further shown in FIG. 6, the actuation module 601 comprises an external motor control switch 604 for actuating the tissue collection element 634. The motor control switch 604 activates the drive mechanism which can then actuate the tissue collection element 634 to cause the tissue collection element 634 to rotate, thereby excising tissue. In some embodiments, the tissue collection element 634 is translated through the tissue manually. Alternatively, the motor control switch 604 activates the drive mechanism to actuate the tissue collection element 634 to rotate and translate through the tissue. The quick excursion switch 606 on the handle 602 can further be used to excise small predefined volumes of tissue. The tissue collection element 634 is in communication with the distal end of the actuation module 601. The tissue collection element 634 is connected to the actuation module 601 through a luer adaptor 624. The amount of tissue excised can be controlled using the depth gauge 622. The depth stop 626 together with the depth gauge 622 can be used to set the depth the tissue collection element can penetrate into the tissue to be sampled.

FIG. 7 is an illustration of the device 700 as assembled and ready for use. The motor control switch 704 can be activated once the tissue collection element 734 is positioned proximal to the tissue of interest 798. The activation of the motor control switch 704 on the handle 702 causes the actuation module 701 to affect rotational actuation of the tissue collection element 734, as indicated by the arrow in FIG. 7. The tissue collection element 734 can be translated manually to position the distal end of the tissue collection element 734 in proximity to the tissue of interest. The actuation module 701 can then be activated to cause rotational actuation of the tissue collection element 734 to excise and collect tissue. As the tissue collection element 734 rotates, the tissue collection element 734 can be manually advanced through the tissue to a desired depth. Alternatively, once the device 701 has been manually positioned proximal to the tissue of interest, the activation of the actuation module 701 can cause linear actuation of the tissue collection element 734 as well as rotational actuation of the tissue collection element 734. In some embodiments, the device 700 can further comprise an endoscope 790 to visualize the placement of the tissue collection element 734, as shown in FIG. 7. The biopsy device can be adjusted so that the position of the biopsy device relative to the associated endoscope can accommodate the working length of the endoscope. Additionally, in some embodiments, an external negative pressure module 740 can be connected to the distal end of the actuation module 701. The negative pressure module 740 can create constant negative pressure at the site of the tissue excision by activating the negative pressure module 740. In FIG. 7, the negative pressure module 740 is activated by pulling back on the plunger 748. The negative pressure module 740 can facilitate drawing the excised tissue up the tissue collection element 734 thereby containing the excised tissue within the tissue collection element 734.

FIG. 8 illustrates an exploded view of another embodiment of the device, in which the actuation module comprises a negative pressure module. The device 800 comprises a handle 802 comprising translational and rotational actuation elements, a disposable syringe 842, a disposable catheter module 830 consisting of a rotating tissue collection element 834, a cannula 838, flexible tubing 862 connecting the cannula 838 to the syringe 842, a luer-lock or other suitable connector 860 that interfaces the cannula 838 and rotating tissue collection element 834 with the handle 802. The handle 802 can comprise a rotational module 870 and translational actuation module 880. The reusable rotational actuation module 870 inside the handle 802 can transmit translational actuation to the catheter module 830. The sample is procured by the combined effect of rotating the tissue collection element 834, translating the cannula 838 and the tissue collection element 834, and actuating the syringe 842 to provide aspiration. The handle can be designed so that the user can perform all functions during the biopsy with one hand leaving the other hand free to operate the endoscope. The catheter module 830 can snap onto the bottom of the handle and can be easily removed and discarded after the procedure. In some embodiments, the catheter module comprises a bent tube tissue collection element. In some embodiments, the catheter module comprises a drill-bit tissue collection element. In some embodiments, the syringe is a 10 mL disposable syringe. Alternatively, the syringe can be a 5 mL or a 15 mL syringe. The syringe can be any suitable sized syringe.

FIG. 9A illustrates a cross-section view of the actuation module 901 comprising a negative pressure module 940 as assembled. The handle 902 comprises a syringe 942, and the rotational actuation module 970 and the translational actuation module 980. FIG. 9B illustrates a perspective view of the actuation module 901. The device can be designed for one handed use. The actuation module 901 comprises a handle lever 964 for manually translating the cannula and tissue collection element. Additionally, the handle can comprise a ratchet slider switch 966 for releasing the ratchet mechanism that interfaces with the motor module. The actuation module can further comprise a home position slider switch 967 for actuating the tissue collection element and setting the home position for the motor module can also be included. The actuation module 901 can further comprise an aspiration slider switch 968 for partially releasing a compressed spring, thereby actuating a syringe that provides aspiration through the cannula and the rotating tube. During operation, the cannula and rotating tissue collection element can be translated from within the sheath of the endoscope to the desired location near the solid tumor. This is achieved by repeatedly squeezing the hand-activated handle lever. Sample collection will be iterated from this point, so the set position can be designated as the home position. Also, the motor is actuated and the rotating tissue collection element emerges from the cannula. These three actions can be completed simultaneously by sliding the home position switch to the locked position. Once the device is prepared for sample collection, the syringe can be actuated using the aspiration slider switch 968. Sample can then be collected by repeatedly squeezing the handle. After one pass of collecting the sample, the ratchet slider switch 966 is used to return the cannula and the tissue collection element to the home position. Sample collection is iterated until enough sample has been collected. Once sample collection has been completed, the cannula and the tissue collection element can be retracted into the sheath of the endoscope by unlocking the home position switch 967. The sample is subsequently obtained from the cannula by exerting positive pressure with the syringe and locking the home position switch to actuate the tissue collection element. FIG. 9C is a perspective view of the handle of FIG. 9A as viewed from the bottom. FIG. 9D is a cross-sectional view of the handle.

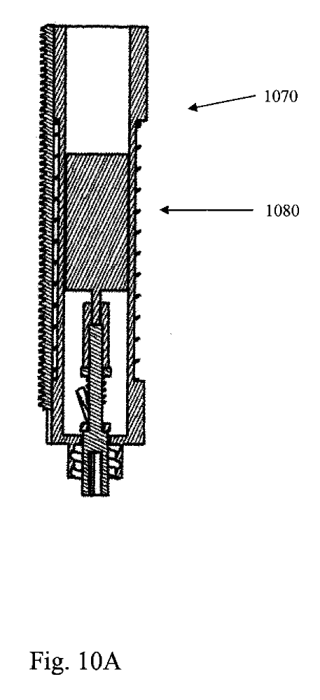

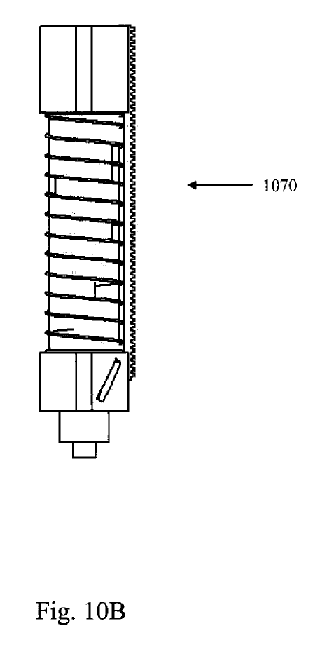

FIG. 10A is a lateral cross-section of the rotational actuation module 1070 and the translational actuation module 1080. FIG. 10B is an isolated view of the rotational actuation module 1070 as viewed from the side.

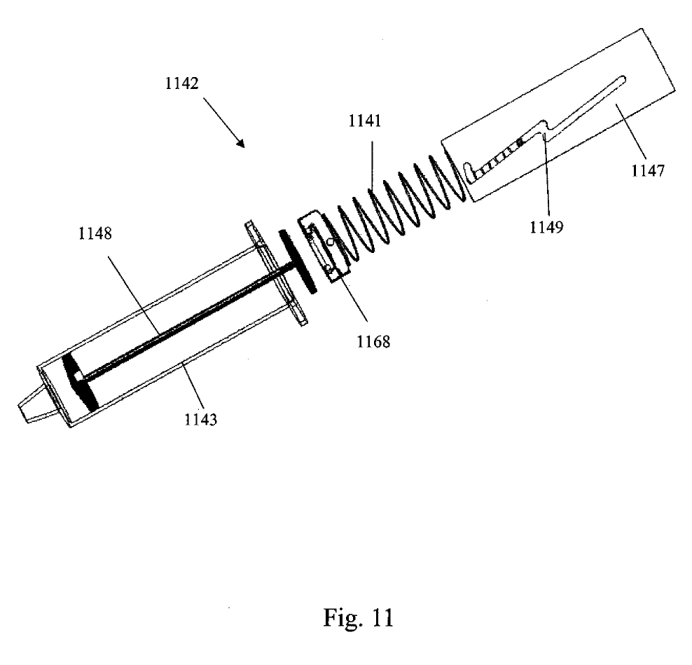

FIG. 11 depicts an exploded view of another embodiment of the syringe 1142. The syringe 1142 comprises a syringe barrel 1143 and a plunger 1148. The syringe 1142 further comprises an aspiration slider switch 1168, a spring 1141, and a negative pressure gauge 1147. The aspiration switch 1168 is in communication with the plunger 1148. The aspiration switch 1168is drawn up through the groove 1149 on the negative pressure gauge 1147. The movement of the aspiration switch 1168 creates negative pressure in the syringe barrel 1143. The negative pressure in the syringe barrel 1143 can be transferred to the tissue collection element and to the portion of tissue to be excised.

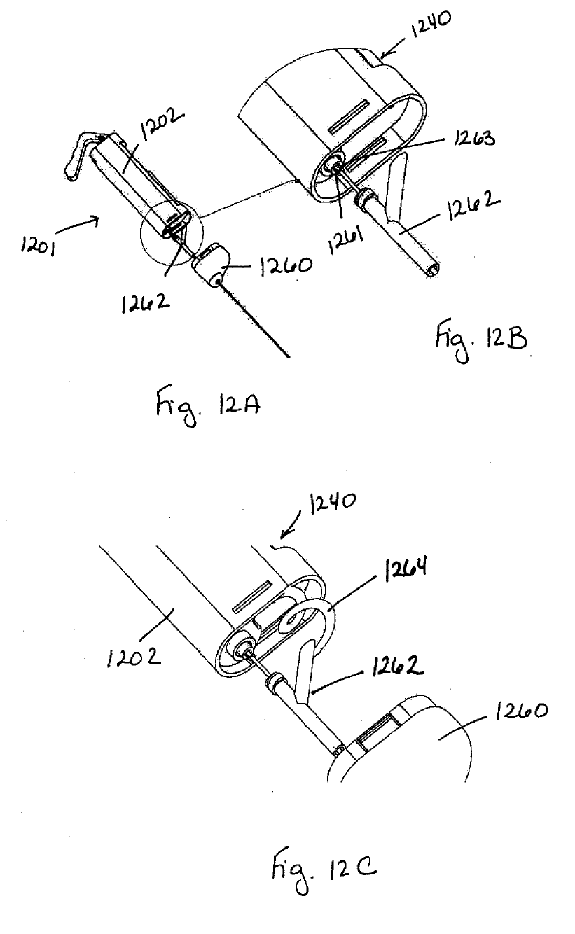

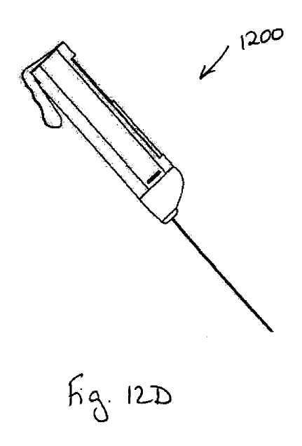

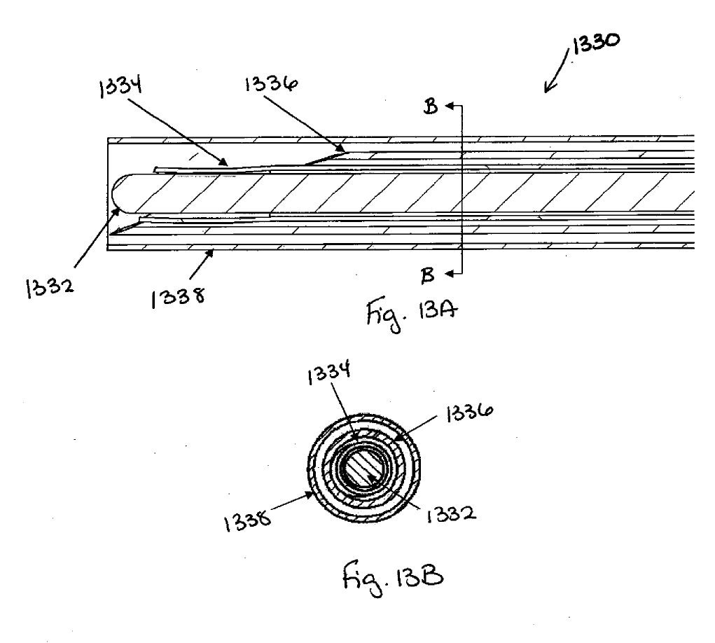

FIGS. 12A-D illustrates the steps for preparing the device for operation. FIG. 12A illustrates how the handle 1202 of the actuation module 1201 can be fit with a y-tube connector 1262. A close-up view of FIG. 12B illustrates how the y-tube connector 1262 from the actuation module is inserted into a magnetic coupler 1261 and secured to the module by twisting a luer-lock 1263. Once these connections have been made, a negative pressure module can be inserted into the actuation module. The negative pressure module 1240 can then be armed and attached to flexible tubing 1264, which in turn is attached to the y-tube connector 1262, as illustrated in FIG. 12C. The connector 1260 with attached catheter module 1230 can then be connected to the handle to complete the device 1200, as shown in FIG. 12D. In some embodiments, the connector 1260 is snap-fit to the handle 1202. In some embodiments, the connector 1260 is screwed to the handle 1202.

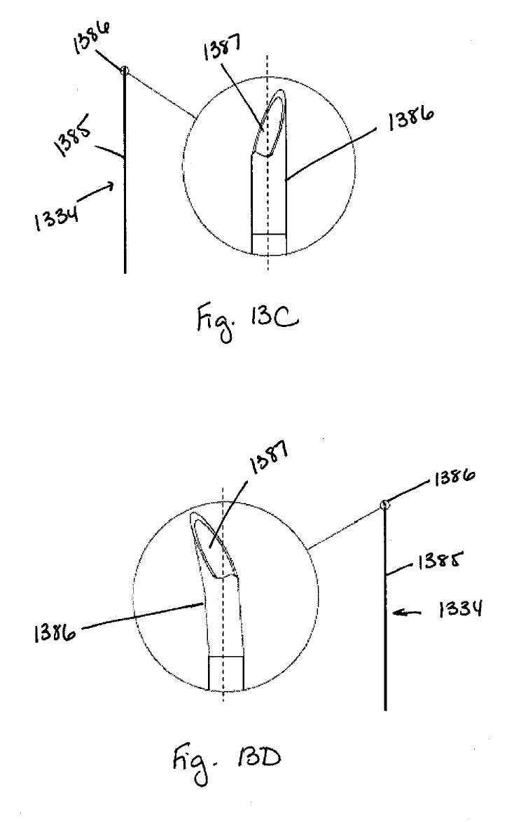

Further provided herein are alternate embodiments of the tissue collection element and the catheter module. FIG. 13A is one embodiment of the catheter module 1330 of a biopsy device described herein. FIG. 13A illustrates a lateral cross-sectional view of the catheter module 1330. The catheter module 1330 comprises a stylet 1332, tissue collection element 1334, and outer needle 1336 nested in a cannula 1338. FIG. 13B illustrates a cross-sectional view of FIG. 13A along the line B-B. FIG. 13C illustrates an isolated tissue collection element 1334. The tissue collection element 1334 comprises a shaft 1385 and a distal end 1386. The distal end 1386 of the tissue collection comprises an opening 1387. The opening 1387 is created such that a larger area of tissue can be cut. When the tissue collection element is introduced proximal to the tissue to be sampled, the tissue collection element can be housed in an outer needle. When housed in an outer needle, the tissue collection element conforms to the shape of the outer needle. Once the outer needle is positioned adjacent to the tissue, the tissue collection element is extended past the distal end of the outer needle. Once the distal end of the tissue collection extend past the distal end of the outer needle, the tissue collection element assumes a secondary configuration where the distal end of the tissue collection element biases away from the center axis of the tissue collection element, as shown in FIG. 13D. In some embodiments, the tissue collection element is held in a first configuration by an outer needle. The tissue collection element can be held in a first configuration by a stylet extending through the tissue collection element. Once the stylet is removed from distal end of the tissue collection element, the tissue collection element can change configuration to a second configuration.

In FIG. 13E, the stylet 1332 is nested in the tissue collection element 1334. The tissue collection element 1334 is nested at least partially in the outer needle 1336. The stylet 1332, tissue collection element 1334, and outer needle 1336 extend past the distal end of the cannula 1338. The components of the catheter module 1330 are nested when the device is introduced to the patient and into the stomach. FIG. 13F is a cross-section of the FIG. 13E along the line F-F, illustrating the nesting of the stylet 1332, the tissue collection element 1334, the outer needle 1336 and the cannula 1338. FIG. 13G is a cross-sectional view of FIG. 13E along the line G-G illustrating the nesting of the stylet 1332, tissue collection element 1334, and outer needle 1336.

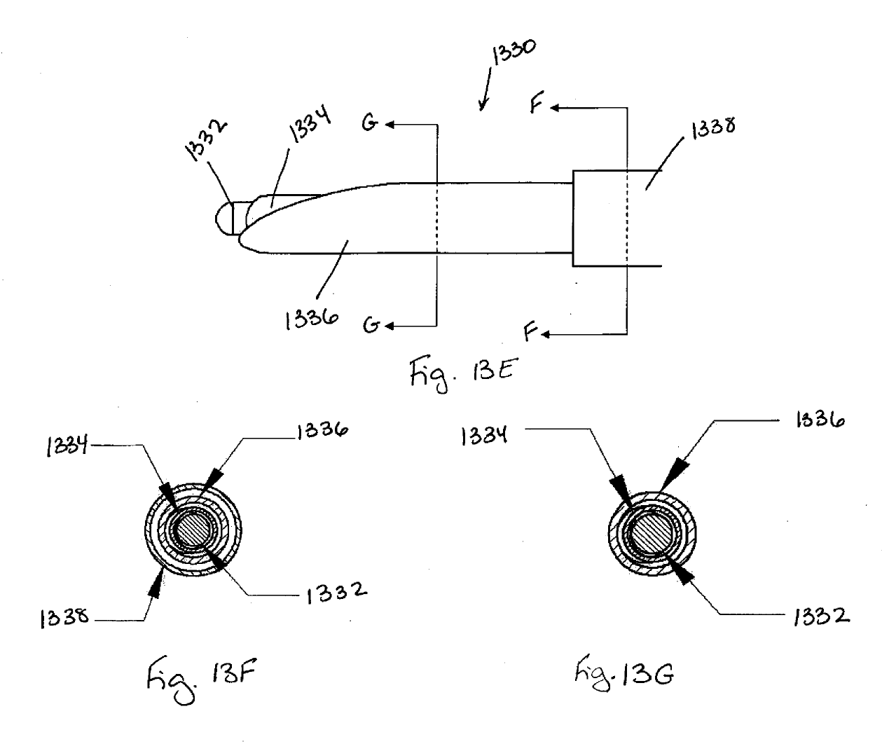

In some embodiments of a biopsy device comprising a bent tube tissue collection element, the catheter module 1430 does not comprise an outer needle, as shown in FIG. 14A. FIG. 14A is a side view of the catheter module 1430 in which the tissue collection element 1434 and stylet 1432 extend past the distal end of the cannula 1438. FIG. 14B is a cross-sectional view of the FIG. 14A along the line B-B. As shown in FIG. 14B, the stylet 1432 is encompassed by the tissue collection element 1434, and both the stylet 1432 and the tissue collection element 1434 are encompassed by the cannula 1438. FIG. 14C is a cross-sectional view of FIG. 14A along the line C-C illustrating the tissue collection element 1434 encompassing the stylet 1432.

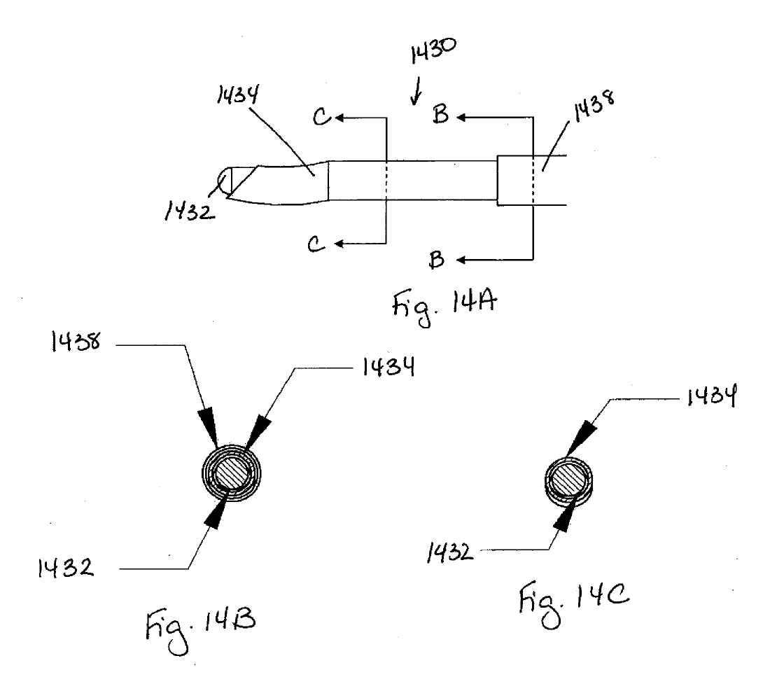

Other embodiments of the tissue collection element are also considered. In some embodiments, the tissue collection element 1534 comprises a twisted configuration. The twisting of the tissue collection element 1534 is similar to a drill-bit. FIG. 15A illustrates a drill-bit tissue collection element 1534. The drill-bit has a structure that is twisted about a center axis. When the drill-bit tissue collection element 1534 is activated the drill-bit rotates about the center axis. When placed in contact with the tissue to be excised, the cutting edges 1552 of the drill-bit cut the tissue, and the tissue is drawn up the grooves 1554 of the drill-bit tissue collection element 1534. As shown in FIG. 15B, the drill-bit tissue collection element 1534 can be encompassed by an outer needle 1536 and a cannula 1538. FIG. 15B is a lateral cross-section of the drill-bit tissue collection element 1534 and catheter module 1530 illustrating the tissue collection element 1534 nested in an outer needle 1536, nesting in a cannula 1538. FIG. 15C is a side view of the catheter module 1530 with a tissue collection element 1534 and outer needle 1536 extending past the distal end of the cannula 1538. FIG. 15D is a cross-sectional view of FIG. 15C along the line D-D, illustrating the cannula 1538 encompassing the outer needle 1536 which further encompasses the tissue collection element 1534. FIG. 15E is a lateral cross of the catheter module 1530 of FIG. 15C with outer needle 1536 encompassing a drill-bit tissue collection element 1534 along the line E-E.

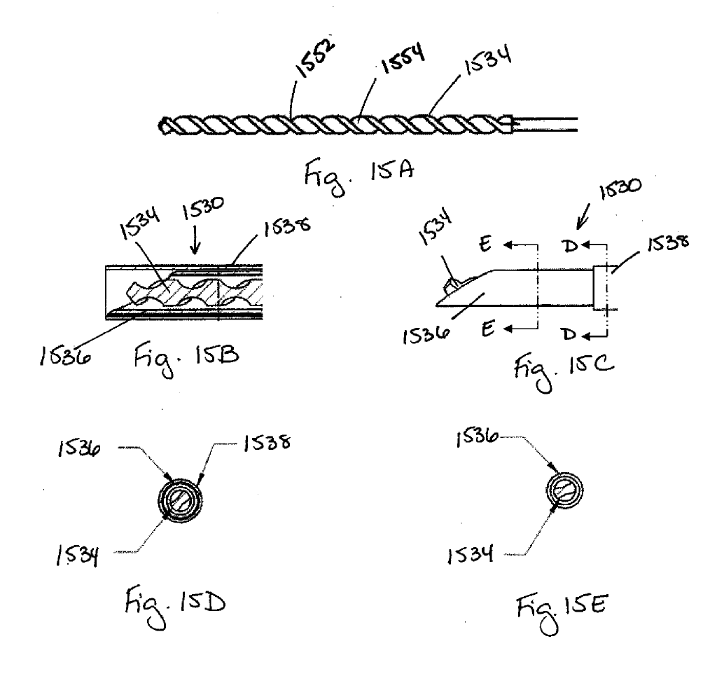

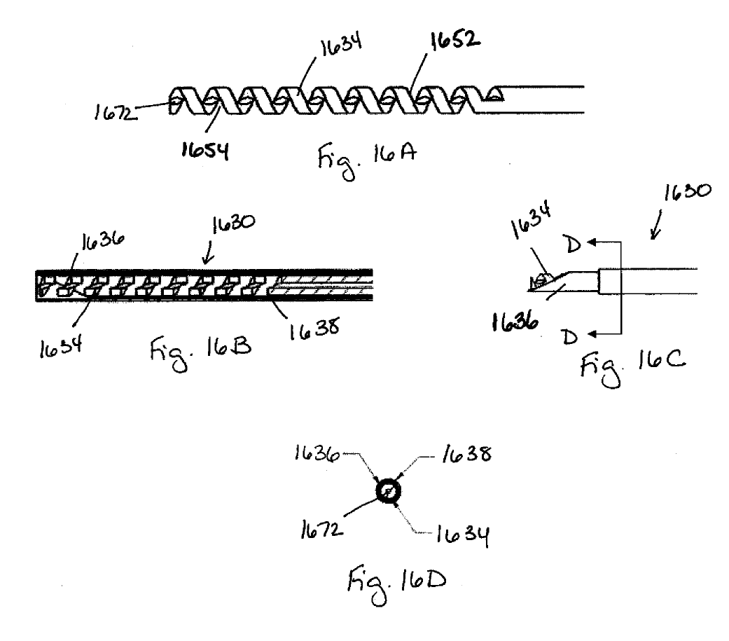

Another embodiment of the biopsy device comprises a drill-bit tissue collection element in the shape of a helical coil with a hollow center. FIG. 16A is an alternative drill-bit tissue collection element 1634 as viewed from the side. The cutting edges 1652 of the drill-bit tissue collection element 1634 rotate when actuated by the drive mechanism of the actuation module. As the drill-bit tissue collection element 1634 is translated through the tissue, a segment of tissue is excised and drawn up the grooves 1654 of the tissue collection element 1634. Alternatively, tissue can be drawn up the center 1672 of the drill bit tissue collection element 1634. FIG. 16B is a lateral cross-sectional view of the catheter module 1630 illustrating the tissue collection element 1634 nested in an outer needle 1636 encompassed by a cannula 1638. FIG. 16C is a side-view of the catheter module 1630 where the outer needle 1636 and the tissue collection element 1634 extend past the distal end of the cannula 1638. FIG. 16D is a cross-sectional view of FIG. 16C along the line D-D, illustrating the nesting of the tissue collection element 1634, the outer needle 1636, and the cannula 1638, and further illustrating the hollow center 1672 of the tissue collection element 1634.

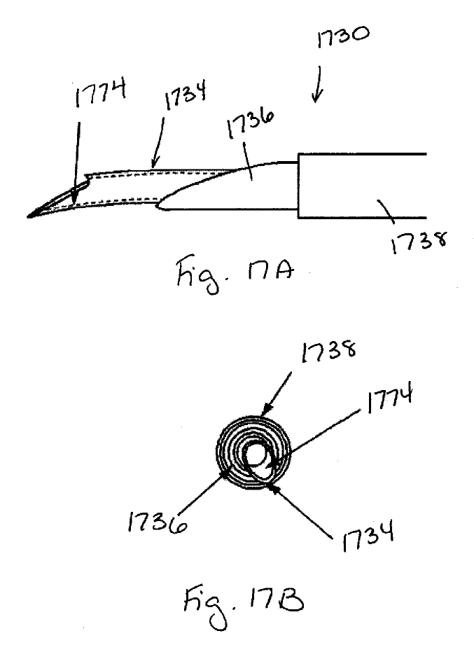

In some embodiments of the invention described herein, the tissue collection element can be coated with a low-friction coating, such as an amorphous fluoropolymer (Teflon® available from Dupont), poly(tetrafluoroethylene), perfluoroalkoxy polymer resin, fluorinated ethylene-propylene, fluoropolymers, and combinations thereof For example, FIG. 17A illustrates one embodiment of a catheter module 1730 in which the tissue collection element 1734 has been coated with a low-friction coating 1774. FIG. 17A is a side view of a bent tube tissue collection element 1734. The outer needle 1736 and tissue collection element 1734 are shown extending past the distal end of the cannula 1738. The tissue collection element 1734 is also shown extending past the distal lend of the outer needle 1736. The tissue collection element 1734 can be coated with a low-friction coating 1774. In some embodiments, the entire tip of the tissue collection element 1734 can be coated with a low friction coating 1774. Alternatively, the inside of the tissue collection element 1734 can be coated with a low-friction coating 1774, as shown in FIG. 17A. Additionally, a low-friction coating can be applied to a drill-bit embodiment of the device. The entire tissue collection element can be coated in a low-friction coating. Alternatively, the low friction coating can be applied to the grooves of the tissue collection element. FIG. 17B is frontal view of the catheter module of FIG. 17A.

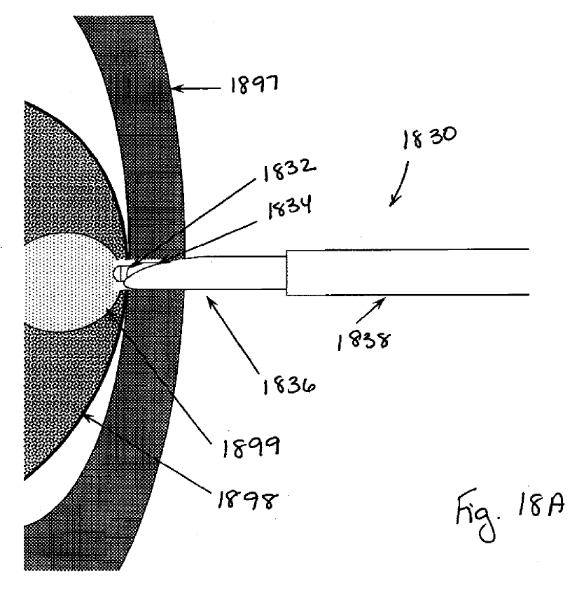

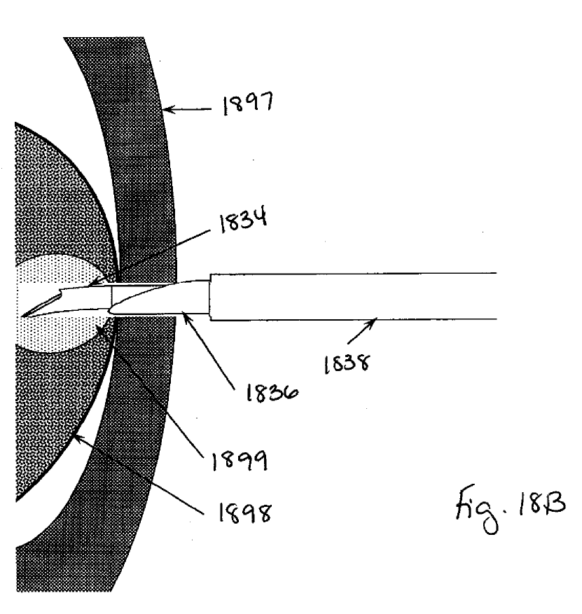

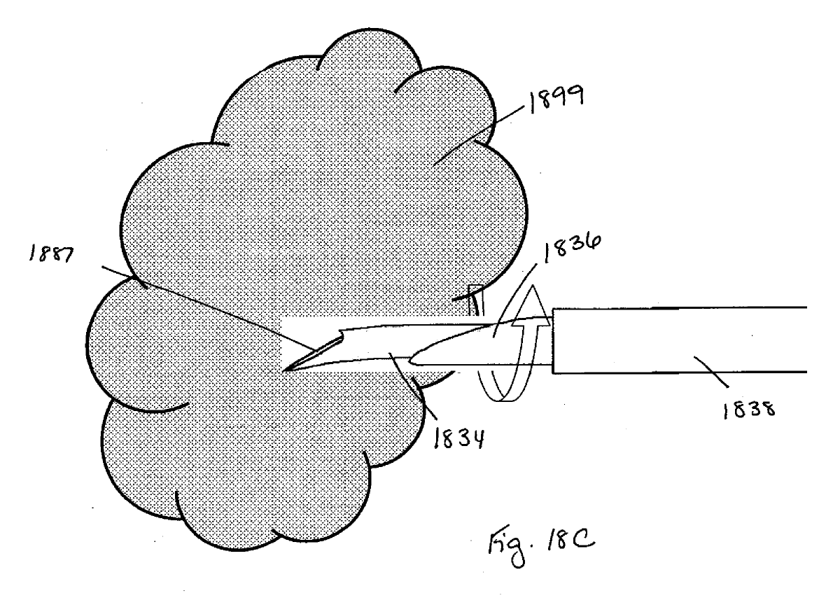

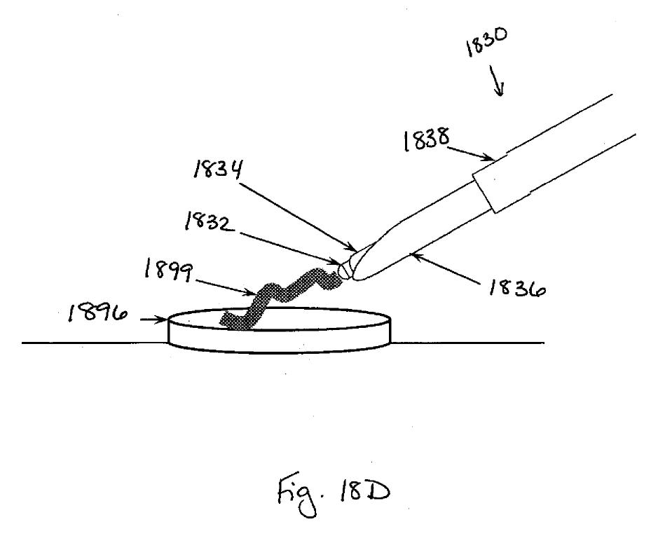

FIGS. 18A-D illustrate one embodiment of a biopsy device in operation. FIG. 18A illustrates the device penetrating the stomach wall. In the embodiment of device shown in FIG. 18A, the catheter module 1830 comprises a cannula 1838, an outer needle 1836, and a tissue collection element 1834. In some embodiments, the device can further comprise a stylet 1832. The stylet 1832 can be used to establish and puncture through the point of penetration of the stomach wall 1897. The outer needle 1836, tissue collection element 1834, and stylet 1832 extend from the distal end of the cannula 1838. The outer needle 1836 facilitates the penetration of the stomach wall 1897 to position the tissue collection element 1834 proximal to the tissue of interest 1898. In FIG. 18A, the catheter module 1830 is positioned proximal to a lesion 1899 in the pancreas 1898. FIG. 18B illustrates how the tissue collection element 1834 is then extended past the distal end of the outer needle 1836. The stylet 1832 is retracted from the tissue collection element 1834 so that tissue can be collected by the tissue collection element 1834. The actuation module can then be activated, thereby causing the tissue collection element 1834 to rotate. In some embodiments, the actuation module facilitates the linear translation of the tissue collection element so that the tissue collection element further extends from the distal end of the outer needle. In some embodiments, negative pressure can be applied through the tissue collection element to draw the excised tissue into the tissue collection element. Alternatively, magnetic forces can be used to draw the excised tissue into the tissue collection element. Any suitable force can be used for drawing excised tissue into the tissue collection element.

FIG. 18C illustrates the device and tissue collection element 1834 in use. Once the tissue collection element 1834 is proximal to the tissue 1899 to be sampled, the tissue collection element 1834 is actuated by the actuation module. The tissue collection element 1834 thereby rotates to excise a portion of tissue 1899, as indicated by the arrow in FIG. 18C. The tissue collection element 1834 can further be translated through the tissue 1899 to collect more tissue. The enlarged opening 1887 of the tissue collection element 1834, together with the rotation of the tissue collection element 1834, enables the biopsy device to excise a larger portion of tissue. Traditional biopsy devices capture a volume of tissue equal to the area of the opening of the tissue collection element times the depth to which the element is inserted into the tissue. In other words, the volume (V) of tissue excised by traditional tissue collection element is the area (A) of the opening multiplied by the depth or length (L) or V=A·L. Making the opening of the tissue collection element elongated and rotating the tissue collection element, instead of moving it along a straight path, can thereby increase the volume (V) of the sample collected while using a tissue collection element of the same diameter as used in the typical case. For example purposes only, the volume of the tissue excised can be described as follows. As compared to current embodiments of biopsy devices, the area of the opening—as perceived from a line of sight tangent to the cutting path—of a device described herein can be described by kA, where A is the area of the catheter multiplied by a constant, k. The device is then actuated so that the opening rotates a desired number of turns through the tissue. The distance that the device travels can be described as L·\sqrt{1+(2πRn)²}, where L is the depth to which the device is inserted, R is the radius of rotation measured as the radial separation between the rotational axis and the centroid of the area kA—through the sample, and 1/n is the distance between each rotation. Therefore, using an embodiment of a device described herein allows you to excise a portion of sample having a volume (V), where V=kA·L·\sqrt{(2πRn)²}, assuming the helical cavity created by the tissue collection element is not self-overlapping.

Once it is decided that a desired amount of tissue has been collected from the lesion or tissue sample, the actuation module is deactivated. The tissue collection element can be retracted into the outer needle and the entire catheter module removed from the patient. In some embodiments, the tissue collection element can be retracted into the cannula and the entire catheter module is removed from the patient. In some embodiments, the catheter module is removed from the patient without retracting the tissue collection element into the outer needle. Once the catheter module is outside the patient, the excised tissue sample can be tested. In some embodiments, the tissue sample is removed from the biopsy device. FIG. 18D illustrates one embodiment of how the tissue sample collected by the tissue collection element 1834 can be removed from the catheter module 1830 of the biopsy device and collected in a suitable container 1896, such as a Petri dish. One way a sample can be removed from the catheter module includes removing the negative pressure and retracting the tissue collection element 1834 into the outer needle 1836. In some embodiments, the negative pressure can be reduced instead of removed. The stylet 1832 can be advanced through the tissue collection element 1834 to expel the excised tissue 1899, by pushing the excised tissue 1899 out of the distal end of the tissue collection element 1834. Alternatively, the tissue collection element 1834 can be retracted to expel the excised tissue. As the tissue collection element 1834 is retracted the tissue is retracted with the tissue collection element 1834 until the tissue contacts the stylet 1832 in the tissue collection element 1834. As the tissue collection element 1834 continues to be withdrawn, the stylet 1832 holds the tissue in position until the tissue collection element 1834 no longer encompasses the tissue. In some embodiments, positive pressure can be applied through the tissue collection element 1834 to expel the excised portion of tissue. In some embodiments, another device can be used to remove the excised portion of tissue. The excised portion of tissue can be tested by removing the excised tissue from the tissue collection element. Alternatively, the tissue can be tested without removing the excised portion of tissue from the tissue collection element. In some embodiments, a testing device is directly connected to the catheter module. Alternatively, a testing element is connected to the entire biopsy device. In some embodiments, the actuation module further comprises a testing element for testing the excised portion of tissue.

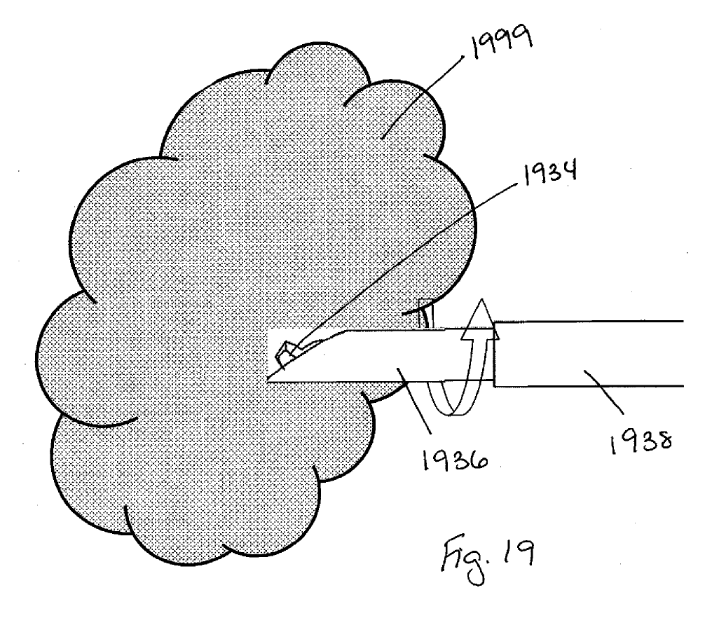

FIG. 19 illustrates another embodiment of the tissue collection element 1934 in use. Once the cannula 1938, outer needle 1936, and tissue collection element 1934 are proximal to the tissue 1999 to be sampled, the tissue collection element 1934 is actuated by the drive mechanism. The tissue collection element thereby rotates (as indicated by the arrow) to excise a portion of tissue. The tissue collection element can further be translated through the tissue to collect more tissue. As the tissue collection element is translated through the tissue sample a spiral shaped segment of tissue can be carved from the sample. As the tissue is cut by the tissue collection element, the tissue moves along the grooves of the tissue collection element and into the cannula. In some embodiments, the excised tissue moves into the cannula through the center of the drill-bit. Alternatively, the excised tissue is drawn into the cannula by following the grooves of drill-bit tissue collection element and through the center of the drill-bit tissue collection element. The tissue can be removed from the cannula module by any suitable means for removing the sample by, for example purposes only, pushing the sample out of the outer needle using the tissue collection element.

### II. Methods

Also provided herein are methods for obtaining biopsy samples. In some embodiments, the method provides for obtaining a measurable target tissue from a collection region comprising: inserting a biopsy device comprising an outer needle having a proximal end and a distal end wherein the proximal end is connected to a drive mechanism, and a tissue collection element formed from material having a first constrained configuration when positioned within the outer needle prior to deployment and a second unconstrained configuration when extended distally beyond the distal end of the outer needle wherein the tissue collection element is translationally and rotationally moveable within the outer needle and distally beyond the distal end of the outer needle in response to actuation by the drive mechanism; advancing the tissue collection element into a patient toward a target tissue; excising a measurable amount of target tissue with the tissue collection element; and removing the excised target tissue from the patient. Additionally, the method can further comprise the step of transmitting a translational actuation force to at least one of the outer needle and the tissue collection element. In some embodiments, the method can further comprise the step of transmitting a rotational actuation force to the tissue collection element. The excising step can further comprises procuring a tissue sample by rotating the tissue collection element while translating the outer needle and tissue collection element. In some embodiments, the method can further comprise the step of step of inserting a stylet into the tissue collection element. Furthermore, the method can further comprise the step of applying negative pressure to the distal tip of at least one of the tissue collection element and cannula. Also, the method can further comprise the step of approaching the target location with the stylet inserted in the tissue collection element prior to sample acquisition.

In some embodiments, a method for obtaining a measurable target tissue from a collection region is provided herein, the method comprising: inserting a biopsy device comprising a stylet having a proximal end and a distal end wherein the stylet is insertable inside the tissue collection element; and a tissue collection element formed from material having a first constrained configuration when the stylet is inserted inside said tissue collection element and a second unconstrained configuration when the stylet is retracted from within the tissue collection element wherein the tissue collection element is translationally and rotationally moveable in response to actuation by the drive mechanism; advancing the tissue collection element into a patient toward a target tissue; excising a measurable amount of target tissue with the tissue collection element; and removing the excised target tissue from the patient. In some embodiments, the method can further comprise the step of transmitting a translational actuation force to the tissue collection element. Alternatively, the method can comprise the step of transmitting a rotational actuation force to the tissue collection element. In some embodiments of the method, the excising step further comprises procuring a tissue sample by rotating the tissue collection element while translating the tissue collection element. The excised tissue can further be removed from the biopsy device. The stylet can be used to remove the excised tissue. In some embodiments, the method can include the application of negative pressure to the distal tip of the tissue collection element. The stylet in the tissue collection element can also be used to approach the target location prior to sample acquisition.

Another method provided herein, is a method for obtaining a target tissue from a collection region comprising: inserting a biopsy device comprising a cannula having a proximal end and a distal end wherein the proximal end is connectable to a drive mechanism; and a tissue collection element having helical cutting features along a portion of its length at the distal end thereof wherein the tissue collection element is adapted to cut target tissue from a collection region and is translationally and rotationally moveable within the outer needle and distally beyond the distal end of the outer needle in response to actuation by the drive mechanism; and a non-friction coating applied to some portion of the distal tip of the tissue collection element and/or the outer needle; advancing the tissue collection element into a patient toward a target tissue; excising a measurable amount of target tissue with the tissue collection element; and removing the target tissue from the patient. In some embodiments, the method can further comprise the step of transmitting a translational actuation force to the cannula and/or tissue collection element. Alternatively, the method can further comprise the step of transmitting a rotational actuation force to the tissue collection element. Furthermore, the excising step further comprises procuring a tissue sample by rotating the tissue collection element while translating the outer needle and tissue collection element. In some embodiments, the negative pressure can be applied to the distal end of at least one of the tissue collection element or outer needle prior to sample acquisition. The method can also provide for the step of approaching the target location with the stylet inserted in the outer needle or tissue collection element prior to sample acquisition.

### III. Kits

Further provided herein is a kit for obtaining a measurable target tissue from a collection region comprising: a removable handle containing a drive mechanism; one or more outer needles, each outer needle having a proximal end and a distal end wherein the proximal end is adaptable to engaging the drive mechanism; and one or more tissue collection elements, each tissue collection element having an adapted and configured form to receive a measurable target tissue from a collection region wherein the tissue collection element is translationally and rotationally moveable within the outer needle distally beyond the distal end of the outer needle in response to the drive mechanism.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A biopsy device comprising:
an outer needle having a distal end and a proximal end connected to a drive mechanism; and
a tissue collection element having a distal end and a proximal end, the tissue collection element formed from material having a first constrained configuration when positioned within the outer needle prior to deployment and a second unconstrained configuration when extended distally beyond the distal end of the outer needle,
wherein the tissue collection element is translationally and rotationally moveable within the outer needle and distally beyond the distal end of the outer needle in response to actuation by the drive mechanism.

2. The biopsy device of claim 1 wherein the distal end of the tissue collection element deviates from the central axis of the outer needle from about 0 degrees to about 180 degrees and rotates around the central axis of the outer needle from about 0 degrees to about 360 degrees.

3. The biopsy device of claim 1 wherein the distal end of the tissue collection element deviates from a central axis of the outer needle along an angle, radius, helical path, or contour.

4. The biopsy device of claim 1 wherein the distal end of the tissue collection element comprises an opening, wherein the opening obtains a portion of tissue having a cross-sectional diameter greater than the cross-sectional diameter of the outer needle.

5. The biopsy device of claim 1 wherein the tissue collection element is translated from within the outer needle to a target location.

6. The biopsy device of claim 1 wherein the distal end of the tissue collection element is rotationally actuated to produce a rotational motion.

7. The biopsy device of claim 6 wherein the rotational motion is a motion selected from the group consisting of continuous, intermittent, reciprocating, and combinations thereof.

8. The biopsy device of claim 1 wherein the tissue collection element is adaptable to be moved manually.

9. The biopsy device of claim 1 wherein the tissue collection element is adaptable to be moved automatically or semi-automatically.

10. The biopsy device of claim 1 wherein the outer needle is adaptable to be moved manually.

11. The biopsy device of claim 1 wherein the outer needle is adaptable to be moved automatically or semi-automatically.

12. The biopsy device of claim 1 wherein the tissue collection element comprises stainless steel.

13. The biopsy device of claim 1 wherein at least a portion of the tissue collection element comprises a shape memory alloy.

14. The biopsy device of claim 1 wherein at least a portion of the tissue collection element is coated with a non-friction coating.

15. The biopsy device of claim 1 wherein at least a portion of the distal end of the outer needle is coated with a non-friction coating.

16. The biopsy device of claim 1 wherein the distal end of the tissue collection element comprises a beveled cutting edge.

17. The biopsy device of claim 1 wherein the tissue collection element is disposable.

18. The biopsy device of claim 1 wherein the tissue collection element cuts and receives tissue within the tissue collection element without further damaging the tissue.

19. The biopsy device of claim 1 wherein the device further comprises a stylet adaptable to be inserted into the tissue collection element.

20. The biopsy device of claim 1 further comprising a negative pressure source adaptable to facilitate application of negative pressure to the distal end of the tissue collection element.

21. The biopsy device of claim 20 wherein the negative pressure is supplied by a syringe.

22. The biopsy device of claim 21 wherein the syringe is a two-stage or a multi-stage syringe.

23. The biopsy device of claim 1 wherein the outer needle is adaptable to be echogenic.

24. The biopsy device of claim 23 wherein the echogenecity is facilitated by rotational actuation applied to the outer needle.

25. The biopsy device of claim 23 wherein echogenecity is facilitated by vibrations induced at the distal tip of the outer needle.

26. The biopsy device of claim 1 wherein the tissue collection element is adaptable to be echogenic.

27. The biopsy device of claim 26 wherein the echogenecity is facilitated by rotational actuation applied to the tissue collection element.

28. The biopsy device of claim 26 wherein the echogenecity is facilitated by vibrations induced at the distal end of the tissue collection element.

29. The biopsy device of claim 1 further comprising a cannula wherein the cannula is adaptable to contain the tissue collection element and outer needle and wherein the tissue collection element and outer needle are translatable and rotatable relative to the cannula.

30. The biopsy device of claim 1 further comprising a depth gauge adaptable to assess the depth of penetration of the tissue collection element within a target location.

31. The biopsy device of claim 1 further comprising a depth stop adaptable to be set to limit the depth of penetration of the tissue collection element within a target location.

32. The biopsy device of claim 1 wherein the tissue collection element is adaptable to capture a measurable target tissue sample from a collection region in at least three passes.

33. The biopsy device of claim 1 further comprising one or more radiopaque markers on at least a portion of the length of the device.

34. The biopsy device of claim 1 wherein the device is adaptable to be operated using single-hand operation.

35. The device of claim 1 further comprising a quick excursion element adaptable to repeatedly extrude depth limited portions of target tissue.

36. The biopsy device of claim 1 wherein the outer needle has a gauge of 18 to 27.

37. The biopsy device of claim 1 wherein the distal end of the tissue collection element extracts a portion of target tissue from a collection region having a diameter greater than 0.05 inches in diameter.

38. The biopsy device of claim 1 further comprising an endoscope having a working length.

39. The biopsy device of claim 38 wherein the biopsy device is adaptable to accommodate the working length of the endoscope.

40. A biopsy device comprising
a stylet having a proximal end and a distal end wherein the stylet is adaptable to be inserted inside the tissue collection element; and
a tissue collection element formed from material having a first constrained configuration when the stylet is inserted inside said tissue collection element and a second unconstrained configuration when the stylet is retracted from within the tissue collection element,
wherein the tissue collection element is translationally and rotationally moveable in response to actuation by the drive mechanism.

41. The biopsy device of claim 40 wherein the distal tip of the tissue collection element deviates from the axis of rotation of said tissue collection element from 0 degrees to 180 degrees and rotates around said axis of rotation from 0 degrees to about 360 degrees.

42. The biopsy device of claim 40 wherein the distal end of the tissue collection element deviates from the axis of rotation of said tissue collection element along an angle, radius, helical path, or contour.

43. The biopsy device of claim 40 wherein the distal end of the tissue collection element comprises an opening wherein the opening obtains a portion of tissue having a cross-sectional diameter greater than the cross-sectional diameter of the tissue collection element in its constrained configuration.

44. The biopsy device of claim 40 wherein the tissue collection element is translated to a target location during tissue acquisition.

45. The biopsy device of claim 40 wherein the distal end of the tissue collection element is rotationally actuated to produce a rotational motion.

46. The biopsy device of claim 45 wherein the rotational motion is a motion selected from the group consisting of continuous, intermittent, reciprocating, and combinations thereof.

47. The biopsy device of claim 40 wherein the tissue collection element is adaptable to be moved manually.

48. The biopsy device of claim 40 wherein the tissue collection element is adaptable to be moved automatically or semi-automatically.

49. The biopsy device of claim 40 wherein the tissue collection element comprises stainless steel.

50. The biopsy device of claim 40 wherein at least a portion of the tissue collection element comprises a shape memory alloy.

51. The biopsy device of claim 40 wherein at least a portion of the distal end of the tissue collection element is coated with a non-friction coating.

52. The biopsy device of claim 40 wherein the distal end of the tissue collection element comprises a beveled cutting edge.

53. The biopsy device of claim 40 wherein the tissue collection element is disposable.

54. The biopsy device of claim 40 wherein the tissue collection element cuts and receives tissue within the tissue collection element without further damaging the tissue.

55. The biopsy device of claim 40 wherein the stylet is adaptable to penetrate tissue as the tissue collection element is advanced toward the target location.

56. The biopsy device of claim 40 wherein the stylet is adaptable to preclude anomalous tissue acquisition as the tissue collection element is advanced toward the target location.

57. The biopsy device of claim 40 wherein the stylet is adaptable to expel a biopsy tissue sample from the tissue collection element.

58. The biopsy device of claim 40 further comprising a negative pressure source adaptable to facilitate application of negative pressure to the distal tip of the tissue collection element.

59. The biopsy device of claim 58 wherein the negative pressure source is a syringe.

60. The biopsy device of claim 59 wherein the syringe is selected from a two-stage or a multi-stage syringe.

61. The biopsy device of claim 40 wherein the tissue collection element is adaptable to be echogenic.

62. The biopsy device of claim 40 wherein the echogenecity is facilitated by rotational actuation applied to the tissue collection element.

63. The biopsy device of claim 40 wherein the echogenecity is facilitated by vibrations induced at the distal end of the tissue collection element.

64. The biopsy device of claim 40 further comprising a cannula adaptable to contain the tissue collection element, the cannula further translatable and rotatable relative to the tissue collection element.

65. The biopsy device of claim 40 further comprising a depth gauge adaptable to assess the depth of penetration of the tissue collection element within a target location.

66. The biopsy device of claim 40 farther comprising a depth stop or similar means for setting a limit for the depth of penetration of the tissue collection element within a target location.

67. The biopsy device of claim 40 wherein the tissue collection element is adaptable to capture a measurable target tissue sample from a collection region in at most three passes.

68. The biopsy device of claim 40 further comprising one or more radiopaque markers on at least a portion of the length of the device.

69. The biopsy device of claim 40 wherein the device is adaptable to be operated using single-hand operation.

70. The biopsy device of claim 40 further comprising a quick excursion element adaptable to repeatedly extrude depth limited-portions of target tissue.

71. The biopsy device of claim 40 wherein the tissue collection element comprises a hollow shaft having a gauge of 18 to 27.

72. The biopsy device of claim 40 wherein the distal end of the tissue collection element is adaptable to extract a portion of target tissue from a collection region having a diameter greater than 0.05 inches in diameter.

73. The biopsy device of claim 40 further comprising an endoscope having a working length.

74. The biopsy device of claim 73 wherein the biopsy device is adaptable to accommodate the working length of the endoscope.

75. A biopsy device comprising:
an outer needle having a proximal end and a distal end wherein the proximal end is connectable to a drive mechanism; and
a tissue collection element comprising a proximal end, a distal end, and a helical cutting edge along at least a portion of the length of the tissue collection element, wherein the helical cutting edge is adaptable to cut a portion of tissue from a target location; and
a non-friction coating adaptable to be applied to at least a portion of the distal end of the tissue collection element,
wherein the tissue collection element is translationally and rotationally moveable within the outer needle and distally beyond the distal end of the outer needle in response to actuation by the drive mechanism.

76. The biopsy device of claim 75 further comprising a non-friction coating adaptable to be applied to at least a portion of the outer needle.

77. The biopsy device of claim 75 wherein the helical cutting edge extends radially from a solid core.

78. The biopsy device of claim 75 wherein the helical cutting edge is adaptable to encircle a hollow core.

79. The biopsy device of claim 75 wherein the tissue collection element is translated from within the outer needle to a target location.

80. The biopsy device of claim 75 wherein the distal end of the tissue collection element is rotationally actuated to produce a rotational motion.

81. The biopsy device of claim 80 wherein the rotational motion is a motion selected from the group consisting of continuous, intermittent, reciprocating, and combinations thereof.

82. The biopsy device of claim 75 wherein the tissue collection element is adaptable to be moved manually.

83. The biopsy device of claim 75 wherein the tissue collection element is adaptable to be moved automatically or semi-automatically.

84. The biopsy device of claim 75 wherein the tissue collection element comprises stainless steel.

85. The biopsy device of claim 75 wherein at least a portion of the distal end of the tissue collection element comprises a beveled cutting edge.

86. The biopsy device of claim 75 wherein the tissue collection element is disposable.

87. The biopsy device of claim 75 wherein the tissue collection element cuts and receives tissue within the outer needle without further damaging the tissue.

88. The biopsy device of claim 75 wherein the device further comprises a stylet adaptable to be inserted in at least one of the outer needle and the tissue collection element.

89. The biopsy device of claim 75 further comprising a negative pressure source adaptable to facilitate application of negative pressure to the distal end of at least one of the outer needle or the tissue collection element.

90. The biopsy device of claim 89 wherein the negative pressure source is a syringe.

91. The biopsy device of claim 90 wherein the syringe is a two-stage syringe or a multi-stage syringe.

92 The biopsy device of claim 75 wherein the outer needle is adaptable to be echogenic.

93. The biopsy device of claim 92 wherein the echogenecity is facilitated by rotational actuation applied to the outer needle.

94. The biopsy device of claim 92 wherein enhanced echogenecity is facilitated by vibrations induced at the distal tip of the outer needle.

95. The biopsy device of claim 75 wherein the tissue collection element is adaptable to be echogenic.

96. The biopsy device of claim 95 wherein the echogenecity is facilitated by rotational actuation applied to the tissue collection element.

97. The biopsy device of claim 95 wherein the echogenecity is facilitated by vibrations induced at the distal tip of the tissue collection element.

98. The biopsy device of claim 75 further comprising a cannula wherein the cannula is adaptable to contain the tissue collection element and outer needle and wherein the tissue collection element and outer needle are translatable and rotatable relative to the cannula.

99. The biopsy device of claim 75 further comprising a depth gauge adaptable to assess the depth of penetration of the tissue collection element within a target location.

100. The biopsy device of claim 75 further comprising a depth stop adaptable to be set to limit the depth of penetration of the tissue collection element within a target location.

101. The biopsy device of claim 75 wherein the tissue collection element captures a measurable target tissue sample from a collection region in at least three passes.

102. The biopsy device of claim 75 further comprising an endoscope having a working length.

103. The biopsy device of claim 102 wherein the biopsy device is adaptable to accommodate the working length of the endoscope.

104. A biopsy device comprising
an outer needle having a proximal end and a distal end wherein the proximal end is connected to a drive mechanism; and
a tissue collection element formed from material having a first constrained configuration when positioned within the outer needle prior to deployment and a second unconstrained configuration when extended distally beyond the distal end of the outer needle wherein the tissue collection element is translationally and rotationally moveable within the outer needle and distally beyond the distal end of the outer needle in response to actuation by the drive mechanism, wherein the distal end of said tissue collection element forms an opening adaptable to obtain a target tissue from a collection region, the opening having a cross-sectional diameter greater than the cross-sectional diameter of the outer needle.

105. A biopsy device comprising
a stylet having a proximal end and a distal end wherein the stylet is adaptable to be inserted inside the tissue collection element; and
a tissue collection element having a proximal end and a distal end, the tissue collection element formed from material having a first constrained configuration when the stylet is inserted inside said tissue collection element and a second unconstrained configuration when the stylet is retracted from within the tissue collection element wherein the tissue collection element is translationally and rotationally moveable in response to actuation by the drive mechanism, wherein the distal end of said tissue collection element forms an opening adaptable to obtain a target tissue from a collection region, the opening having a cross-sectional diameter greater than the cross-sectional diameter of the outer needle.

106. A biopsy device comprising
an outer needle having a proximal end and a distal end wherein the proximal end is connected to a drive mechanism;
a tissue collection element formed from material having a first constrained configuration when positioned within the outer needle prior to deployment and a second unconstrained configuration when extended distally beyond the distal end of the outer needle wherein the tissue collection element is translationally and rotationally moveable within the outer needle and distally beyond the distal end of the outer needle in response to actuation by the drive mechanism; and
a non-friction coating.

107. The biopsy device of claim 106 wherein the non-friction coating is adaptable to be applied to the distal end of the tissue collection element.

108. The biopsy device of claim 106 wherein the non-friction coating is adaptable to be applied to the distal end of the outer needle.

109. A biopsy device comprising
a stylet having a proximal end and a distal end wherein the stylet is insertable inside the tissue collection element; and
a tissue collection element formed from material having a first constrained configuration when the stylet is inserted inside said tissue collection element and a second unconstrained configuration when the stylet is retracted from within the tissue collection element wherein the tissue collection element is translationally and rotationally moveable in response to actuation by the drive mechanism; and
a non-friction coating applied to some portion of the distal tip of the tissue collection element.

110. A kit for obtaining a measurable target tissue from a collection region comprising:
a removable handle containing a drive mechanism;
one or more outer needles, each outer needle having a proximal end and a distal end wherein the proximal end is adaptable to engage the drive mechanism; and
one or more tissue collection elements, each tissue collection element having an adapted and configured form to receive a measurable target tissue from a collection region wherein the tissue collection element is translationally and rotationally moveable within the outer needle distally beyond the distal end of the outer needle in response to the drive mechanism.

111. A method for obtaining a measurable target tissue from a collection region comprising:
inserting a biopsy device comprising an outer needle having a proximal end and a distal end wherein the proximal end is connected to a drive mechanism, and a tissue collection element formed from material having a first constrained configuration when positioned within the outer needle prior to deployment and a second unconstrained configuration when extended distally beyond the distal end of the outer needle wherein the tissue collection element is translationally and rotationally moveable within the outer needle and distally beyond the distal end of the outer needle in response to actuation by the drive mechanism;
advancing the tissue collection element into a patient toward a target location;
excising a measurable amount of target tissue with the tissue collection element; and
removing the excised target tissue from the patient.

112. The method of claim 111 further comprising the step of transmitting a translational actuation force to at least one of the outer needle and the tissue collection element.

113. The method of claim 111 further comprising the step of transmitting a rotational actuation force to the tissue collection element.

114. The method of claim 111 wherein the excising step further comprises procuring a tissue sample by rotating the tissue collection element while translating the outer needle and tissue collection element.

115. The method of claim 111 further comprising the step of inserting a stylet into the tissue collection element.

116. The method of claim 111 further comprising the step of applying negative pressure to the distal end of at least one of the tissue collection element and outer needle.

117. The method of claim 111 further comprising the step of approaching the target location with the stylet inserted in the tissue collection element prior to sample acquisition.

118. A method for obtaining a measurable target tissue from a collection region comprising:
inserting a biopsy device comprising a stylet having a proximal end and a distal end wherein the stylet is insertable inside the tissue collection element; and a tissue collection element formed from material having a first constrained configuration when the stylet is inserted inside said tissue collection element and a second unconstrained configuration when the stylet is retracted from within the tissue collection element wherein the tissue collection element is translationally and rotationally moveable in response to actuation by the drive mechanism;
advancing the tissue collection element into a patient toward a target location;
excising a measurable amount of target tissue with the tissue collection element; and
removing the excised target tissue from the patient.

119. The method of claim 118 further comprising the step of transmitting a translational actuation force to the tissue collection element.

120. The method of claim 118 further comprising the step of transmitting a rotational actuation force to the tissue collection element.

121. The method of claim 118 wherein the excising step further comprises procuring a tissue sample by rotating the tissue collection element while translating the tissue collection element.

122. The method of claim 118 further comprising the step of removing the excised tissue from the biopsy device.

123. The method of claim 122 wherein the stylet is adapted to remove the excised tissue.

124. The method of claim 118 further comprising the step of applying negative pressure to the distal tip of the tissue collection element.

125. The method of claim 118 further comprising the step of approaching the target location with the stylet inserted in the tissue collection element prior to sample acquisition.

126. A method for obtaining a target tissue from a collection region comprising:
inserting a biopsy device comprising a outer needle having a proximal end and a distal end wherein the proximal end is connectable to a drive mechanism; and a tissue collection element having helical cutting features along a portion of its length at the distal end thereof wherein the tissue collection element is adapted to cut target tissue from a collection region and is translationally and rotationally moveable within the outer needle and distally beyond the distal end of the outer needle in response to actuation by the drive mechanism; and a non-friction coating applied to some portion of the distal tip of the tissue collection element and/or the outer needle;
advancing the tissue collection element into a patient toward a target location;
excising a measurable amount of target tissue with the tissue collection element; and
removing the target tissue from the patient.

127. The method of claim 126 further comprising the step of transmitting a translational actuation force to the outer needle and/or tissue collection element.

128. The method of claim 126 further comprising the step of transmitting a rotational actuation force to the tissue collection element.

129. The method of claim 126 wherein the excising step further comprises procuring a tissue sample by rotating the tissue collection element while translating the outer needle and tissue collection element.

130. The method of claim 126 further comprising the step of applying negative pressure to the distal tip of the tissue collection element and/or outer needle prior to sample acquisition.

131. The method of claim 126 further comprising the step of approaching the target location with the stylet inserted in the outer needle or tissue collection element prior to sample acquisition.
